(19) United States
(12) Patent Application Publication
(10) Pub. No.: US 2018/0087041 A1
(43) Pub. Date: Mar. 29, 2018

(54) TAGGED FORM OF MUT ENZYME, GENETIC CONSTRUCTS INCORPORATING IT, AND ITS USE IN GENE THEREAPY

(71) Applicant: The United States of America, as represented by the Secretary, Dept. of Health and Human Services, BETHESDA, MD (US)

(72) Inventor: Charles P. Venditti, POTOMAC, MD (US)

(21) Appl. No.: 15/563,379

(22) PCT Filed: Apr. 22, 2016
(86) PCT No.: PCT/US16/28974

§ 371 Date: Sep. 29, 2017

Related U.S. Application Data
(60) Provisional application No. 62/152,520, filed on Apr. 24, 2015.

(51) Int. Cl. C12N 9/90; A61K 48/00; A61K 38/52

(52) U.S. Cl. CPC C12N 9/90; C12Y 504/99002; C07K 2319/20; A61K 38/52; C07K 2319/07; A61K 48/0058

(57) ABSTRACT

Disclosed are polynucleotides, polypeptides, and gene therapy vectors relating to biologically active methylmalonyl-CoA mutase enzymes, internally tagged with an immunoaffinity and detection epitope, which has been designed and tested in mouse models of methylmalonic acidemia (MMA). The polypeptides and polynucleotides of the present invention contain a mitochondrial leader sequence fused to tag, such as an HA, 3xFLAG, or V5 tag placed in a region of the methylmalonyl-CoA mutase enzyme that maintains mitochondrial localization and function, e.g., the 5′ end of a methylmalonyl-CoA mutase polynucleotide is replaced with an engineered nucleotide sequence that encodes the endogenous mitochondrial importation sequence, a mitochondrial protease cleavage site, and a tag. The polynucleotides and polypeptides of the invention are useful to treat conditions such as MMA, and to assay both activity and biodistribution after gene therapy in varied models of MMA.

### CROSS-REFERENCE TO RELATED APPLICATIONS

This application claims the priority benefit of U.S. Provisional Application No. 62/152,520, filed Apr. 24, 2015, all of which is incorporated by reference herein in its entirety.

### STATEMENT OF GOVERNMENT INTEREST

The instant application was made with government support; the government has certain rights in this invention.

### SEQUENCE LISTING

The Sequence Listing text file attached hereto, created Apr. 19, 2016, size 96 kilobytes, and filed herewith as file name “sequence listing PCT_ST25.txt” is incorporated herein by reference in its entirety.

### BACKGROUND

Methylmalonic acidemia (MMA) is an autosomal recessive disorder caused by defects in the mitochondrial localized enzyme methylmalonyl-CoA mutase (MUT) (Manoli, et al. 2010 Methylmalonic Acidemia (in Gene Reviews, eds. Pagon, et al.)). The estimated incidence of MMA is 1 in 25,000-48,000. MUT is an enzyme that catalyzes the conversion of L-methylmalonyl-CoA to succinyl-CoA. This reaction is one of several enzymatic reactions required to metabolize branch chain amino acids, odd chain fatty acids, and propionate produced by the gut flora (Chandler, et al. 2005 Mol Genet Metab 86:34-43). MUT deficiency, the most common cause of MMA, is characterized by the accumulation of methylmalonic acid and other disease-related metabolites. The disease is managed with dietary restriction of amino acid precursors and cofactors but lacks definitive therapy. MMA can lead to metabolic instability, seizures, strokes, and kidney failure, and it can be lethal even when patients are being properly managed, underscoring the need for new therapies for this disease. Even though MMA is rare, all babies born in the USA are screened for this condition as newborns, emphasizing the need to develop better therapies.

The prognosis for long-term survival in MMA patients is poor. This has been established, repeatedly, since the first studies on outcome were published in the early 1980s and still remains dismal more than three decades later: the mortality of Mut MMA was ˜60% or higher in the 1980s and has improved only slightly to ˜40% by the first decade in the 2000s. The unacceptably high mortality experienced by isolated MMA patients has led centers to pursue elective liver and combined liver-kidney transplantation as a treatment for the metabolic instability that eventually causes demise. When successful, solid organ transplantation can eliminate many symptoms of MMA, but has numerous practical limitations that include procedural availability, surgical mortality and morbidity, expense, a finite donor pool, and the need for life-long immune suppression. Therefore, alternative approaches to restore enzyme activity to the liver and other tissues in patients with MMA are clearly needed.

### BRIEF SUMMARY OF THE EMBODIMENTS

Generally, the subject invention includes a gene therapy vector that expresses a biologically active methylmalonyl-CoA mutase enzyme, internally tagged with an immunoaffinity and detection epitope, (e.g., tag-methylmalonyl-CoA mutase fusion enzyme) which has been designed and tested in mouse models of MMA. In one embodiment, the methylmalonyl-CoA mutase fusion enzymes of the present invention contain a mitochondrial leader sequence fused to a tag placed in a region of the methylmalonyl-CoA mutase enzyme that maintains mitochondrial localization and function. In one embodiment, the 5′ end of a Mut or methylmalonyl-CoA mutase gene is replaced with an engineered nucleotide sequence that encodes the endogenous mitochondrial importation sequence, a mitochondrial protease cleavage site, and a tag. One or more linker sequences may flank the tag in order to maintain protein folding and functionality for both the tag and the methylmalonyl-CoA mutase enzyme.

The murine V5-methylmalonyl-CoA mutase fusion enzyme, human HA-methylmalonyl-CoA mutase fusion enzyme, and human 3xFLAG-methylmalonyl-CoA mutase enzyme, in one embodiment were found to have full biological activity in vivo, as well as excellent expression, and the epitope tag allows for facile detection when co-expressed with an endogenous missense mutation of the methylmalonyl-CoA mutase gene (Mut). This genetically engineered, non-naturally occurring DNA sequence, which encodes methylmalonyl-CoA mutase can be used to express biologically functional methylmalonyl-CoA mutase to treat conditions such as MMA, and is useful to assay both activity and biodistribution after AAV or other gene therapy in varied models of MMA.

The tag-Mut or tag-MUT (referring to fusion enzymes incorporating murine or human methylmalonyl-CoA mutase enzyme, respectively) fusion enzyme transgenes of the present invention can be used as a drug, via viral- or non-viral-mediated gene delivery, to restore MUT function in MMA patients, prevent metabolic instability, and ameliorate disease progression. Because this enzyme may also be important in other disorders of branched chain amino acid oxidation, gene delivery of synthetic MUT nucleotides of the present invention could be used to treat conditions other than MUT MMA.

Additionally, the tag-MUT fusion enzyme transgenes of the present invention can be used for the in vitro production of MUT for use in enzyme replacement therapy for MMA. Enzyme replacement therapy is accomplished by administration of the tag-MUT fusion enzymes of the invention orally, sub-cutaneously, intra-muscularly, intravenously, or by other therapeutic delivery routes.

In another application, the tag-Mut or tag-MUT fusion enzyme transgenes of the present invention can be delivered as mRNAs, modified mRNAs, or peptide nucleic acids, either as solitary agents or packaged as nanoparticles, encapsulated with lipid, polymers to enhance tissue specific uptake, such as by the liver, for therapeutic uses and biodistribution studies.

Thus, in one embodiment, the present invention includes a synthetic methylmalonyl-CoA mutase (e.g., MUT, Mut) polypeptide which sequence may comprise, in order from the N-terminus: a methylmalonyl-CoA mutase mitochondrial leader amino acid sequence, a tag amino acid sequence, and a methylmalonyl-CoA mutase mature amino acid sequence. Optionally, the synthetic polypeptide may further comprise at least one linker sequence(s) flanking the tag sequence. The synthetic polypeptide of the invention, in one embodiment, comprises a methylmalonyl-CoA mutase mitochondrial leader amino acid sequence which comprises a human or a mouse mitochondrial leader amino acid sequence which includes SEQ ID NO:1, SEQ ID NO:2, and/or an amino acid sequence having at least about 95% identity thereto, and having substantially identical activity to the methylmalonyl-CoA mutase mitochondrial leader amino acid sequence. The optional linker sequence(s), in one embodiment, may comprise an amino acid sequence of from 1 to about 15 amino acids and optionally may comprise only amino acids selected from the group consisting of serine (S), alanine (A), lysine (K), tyrosine (Y), threonine (T), phenylalanine (F), and glycine (G). Specific examples of suitable linker sequences include MSYY (SEQ ID NO: 3), SKEFGT (SEQ ID NO: 4), GG (SEQ ID NO: 5), GGSS (SEQ ID NO: 6), and/or G (SEQ ID NO: 7).

Synthetic methylmalonyl-CoA mutase(s) of the present invention may also, in one embodiment, comprise an amino acid tag. The tag may comprise, for example, a 3xFLAG tag, an HA tag, a V5 tag, a Myc-tag, a poly-HIS tag, a VSV tag, an Xpress tag, an isopeptag, and/or a spytag, examples of which include SEQ ID NO:8, SEQ ID NO:9, SEQ ID NO:10, SEQ ID NO:11, SEQ ID NO:12, SEQ ID NO:13, SEQ ID NO:14, SEQ ID NO:15, and/or SEQ ID NO:16. In one embodiment, the tag can include SEQ ID NO:8, SEQ ID NO:9, and/or SEQ ID NO:10. In another embodiment, the linker+tag comprises an amino acid sequence that can include SEQ ID NO:36 (GGSSYPYDVPDYAGG), SEQ ID NO:37 (GGSSDYKDDDDKGDYKDDDDKGDYKDDDDKGG), and/or SEQ ID NO:38 (MSYYGKPIPN PLLGLDSTSKEFGT).

The synthetic methylmalonyl-CoA mutase of the present invention may also comprise, in one embodiment, a methylmalonyl-CoA mutase mature amino acid sequence including SEQ ID NO:17, SEQ ID NO:18, and/or an amino acid sequence having at least about 95% identity thereto, and having substantially identical activity to the methylmalonyl-CoA mutase mature amino acid sequence.

Embodiments of a tagged synthetic methylmalonyl-CoA mutase of the present invention include, for example, an amino acid sequence comprising SEQ ID NO:19, SEQ ID NO:20, SEQ ID NO:21, SEQ ID NO:22, SEQ ID NO:23, SEQ ID NO:24, SEQ ID NO:25, SEQ ID NO:26, and/or an amino acid sequence having at least about 95% identity thereto, and having substantially identical activity to the methylmalonyl-CoA mutase mature amino acid sequence.

The present invention also includes synthetic methylmalonyl-CoA mutase (Mut or MUT) polynucleotide which encodes for the polypeptides of the invention. For example, the present invention includes a synthetic polynucleotide which encode embodiments of the tagged methylmalonyl Co-A mutase of the invention, namely, SEQ ID NO:27, SEQ ID NO:28, SEQ ID NO:29, SEQ ID NO:30, and/or a nucleic acid sequence having at least about 95% identity thereto, wherein the synthetic polynucleotide encodes for a polypeptide that has substantially identical activity to WT methylmalonyl-CoA mutase. The polynucleotides of the invention may include a Mut or MUT coding sequence, such as, for example, SEQ ID NO:31, SEQ ID NO:32, SEQ ID NO:33, SEQ ID NO:34, and/or SEQ ID NO:35, and this Mut or MUT coding sequence may further comprise a polynucleotide encoding a tag flanked by linkers inserted between, for example, nucleotides 96 and 97, and a polynucleotide sequence having at least about 95% identity thereto, wherein the synthetic polynucleotide encodes for a polypeptide that has substantially identical activity to WT methylmalonyl-CoA mutase. Tags to include in the synthetic polynucleotides of the invention can include SEQ ID NO:8, SEQ ID NO:9, SEQ ID NO:10, SEQ ID NO:11, SEQ ID NO:12, SEQ ID NO:13, SEQ ID NO:14, SEQ ID NO:15, and/or SEQ ID NO:16, and the optional linkers can comprise at least one polynucleotide encoding the polypeptide selected from the group consisting of MSYY (SEQ ID NO: 3), SKEFGT (SEQ ID NO: 4), GG (SEQ ID NO: 5), GGSS (SEQ ID NO: 6), and/or G (SEQ ID NO: 7); or the tag comprises a polynucleotide such as SEQ ID NO:36, SEQ ID NO:37, and/or SEQ ID NO:38.

In some embodiments, the synthetic polynucleotides of the invention are DNA sequences. In other embodiments, the synthetic polynucleotides of the invention are RNA sequences or other modified forms of either nucleic acid, such as peptide nucleic acids or modified mRNAs. The present invention also includes a recombinant expression vector which comprises the polynucleotides of the present invention, as well as pharmaceutical compositions with polynucleotides and polypeptides of the present invention with a pharmaceutically acceptable carrier.

The present invention also includes a method of treating a disease or condition mediated by methylmalonyl-CoA mutase (MUT), the method comprising administering to a subject in need thereof, a therapeutic amount of a synthetic polynucleotide or synthetic polypeptide of the invention. In one embodiment, the disease or condition is methylmalonic acidemia (MMA).

The method of treating the disease can include gene therapy. Gene therapy can involve in vivo gene therapy (direct introduction of the genetic material into the cell or body) or ex vivo gene transfer into a subject or patient, of the DNA or RNA nucleotides of the present invention, which usually involves genetically altering cells prior to administration, resulting in therapeutically effective amounts of the nucleotides/polypeptides of the present invention in the subject/patient. In another embodiment, genome editing, or genome editing with engineered nucleases (GEEN) may be performed with the nucleotides of the present invention allowing DNA to be inserted, replaced, or removed from a genome using artificially engineered nucleases.

The present invention also includes a method for detecting the expression of a synthetic polynucleotide of the present invention which includes the steps of administering the synthetic polynucleotide in an expression vector to a subject, obtaining a sample of the patient's tissue, and determining the level of expression of the synthetic polynucleotide in the patient's tissue.

The present invention also includes a transgenic animal whose genome comprises the synthetic polynucleotide described herein. In another aspect, the invention is directed to a method for producing such a transgenic animal, comprising: providing an exogenous expression vector comprising a polynucleotide comprising a promoter operably linked to the synthetic polynucleotide described herein; introducing the vector into a fertilized oocyte; and transplanting the oocyte into a female animal. Methods for producing transgenic animals are known in the art and include, without limitation, transforming embryonic stem cells in tissue culture, injecting the transgene into the pronucleus of a fertilized animal egg (DNA microinjection), genetic/genome engineering, viral delivery (for example, retrovirus-mediated gene transfer). Transgenic animals according to the invention include, without limitation, rodent (mouse, rat, squirrel, guinea pig, hamster, beaver, porcupine), frog, ferret, rabbit, chicken, pig, sheep, goat, cow primate, and the like.

### BRIEF DESCRIPTION OF THE DRAWINGS

FIG. 1(A)-1(D) illustrates the muscle specific (Mut^{-/-};Tg^{INS-MCK-Mut}) mouse model. Mut^{-/-};Tg^{INS-MCK-Mut} mice develop hepatorenal mitochondropathy associated with renal insufficiency.

FIG. 1(A) shows a schematic of the Tg^{INS-MCK-Mut} rescue construct. The murine Mut cDNA was cloned behind the full length creatine kinase promoter that was flanked by chicken HS4 insulator elements to create INS-MCK-Mut, which was then introduced into the germline of C57BL/6 mice, and bred to create Mut^{-/-};Tg^{INS-MCK-Mut} mice.

FIG. 1(B) shows electron micrographs of liver samples from a 30 day old littermate (Mut^{+/-}) compared to the liver from a Mut^{-/-}; Tg^{INS-MCK-Mut} experimental. Note the distorted mitochondria, with a pale matrix, and intramitochondrial lipid inclusions.

FIG. 1(C) shows electron micrographs of kidney samples from the same mice. The proximal tubular epithelia contain increased numbers of pale and distorted mitochondria in the Mut^{-/-}; Tg^{INS-MCK-Mut} mice.

FIG. 1(D) shows the glomerular filtration rate measured in Mut^{-/-}; Tg^{INS-MCK-Mut} compared to age and diet matched littermates maintained on a high fat, carbohydrate enriched mouse diet at 90 days of age. A severe reduction in the measured GFR can be appreciated in the mutants compared to controls.

FIG. 2(A)-2(D) illustrate the partial deficiency (Mut^{-/-};Tg^{CBAMutG715V}) mouse model. Mut^{-/-};Tg^{CBAMutG715V} mice display panorgan transgene expression, MMA that is inducible, depressed hepatic glutathione, and ultrastructural evidence of megamitochondria formation after the ingestion of a high protein diet.

FIG. 2(A) shows a schematic of the Tg^{CBAMutG715V} rescue construct. The murine mutation was cloned behind the CMV enhanced, chicken-beta actin promoter and between chicken HS4 insulator elements, then introduced into the germline of C57BL/6 mice, and bred to create Mut^{-/-};Tg^{CBAMutG715V} mice.

FIG. 2(B) shows that Mut^{-/-};Tg^{CBAMutG715V} mice display panorgan transgene expression by Western analysis using anti-Mut antibodies and a complex II control to probe 25 μg of tissue extracts shows wild type or greater expression of the Mut transgene in the major organs involved in MMA.

FIG. 2(C) (left) shows Mut^{-/-};Tg^{CBAMutG715V} mice (n=5) display significant metabolite elevations on a regular diet (average serum methylmalonic acid concentration=225 μM; controls) that elevates to an average of 570 μM when fed a high protein diet. Controls fed either diet (n=6) had average serum methylmalonic acid concentration of 1-2 μM, with no change induced by the diet. (Right) Hepatic glutathione is depressed in Mut^{-/-};Tg^{CBAMutG715V} mice (n=4) compared to control littermates (n=4) when fed a regular mouse diet.

FIG. 2(D) shows electron micrographs of the liver from a Mut^{-/-};Tg^{CBAMutG715V} fed a regular diet compared to the same animals fed a high protein diet for 2 months. The protein challenge produces severe mitochondrial morphological changes, including swelling, loss of the cristae and pallor of the matrix.

FIG. 3(A) shows that treatment of Mut^{-/-};Tg^{INS-MCK-Mut} mice with V5Mut (SEQ ID NO:1) polynucleotide delivered using an AAV (adeno-associated virus) restored the whole body oxidative capacity to metabolize 1-C¹³ labeled propionic acid, which is a direct precursor of methylmalonic acid and unmetabolizeable without the action of MUT in the liver.

FIG. 3(B) shows that V5Mut (SEQ ID NO:1) polynucleotide delivered using an AAV (adeno-associated virus) lowered the levels of plasma methylmalonic acid in the blood.

FIG. 4 shows treatment of Mut^{-/-};Tg^{INS-MCK-Mut} mice with V5Mut polynucleotide delivered using an AAV (adeno-associated virus) restored and improved growth to that seen in wild type mice.

FIG. 5(A) shows that treatment of Mut^{-/-};Tg^{CBAMutG715V} mice with V5Mut (SEQ ID NO:1) polynucleotide delivered using an AAV (adeno-associated virus) improved Mut expression in the liver.

FIG. 5(B) shows Western blot detection of the polypeptide product of V5Mut polynucleotide (SEQ ID NO:1) in the liver of Mut^{-/-};Tg^{CBAMutG715V} mice.

FIG. 6 shows the detection of expression of (HA-synMUT4) (SEQ ID NO:29) and 3XFLAG-synMUT4 methylmalonyl-CoA mutase gene (3XFLAG-synMUT4) (SEQ ID NO:30) in 293T cells, by Western blot.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### Definitions

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices, and materials are now described.

As used in this application, including the appended claims, the singular forms “a,” “an,” and “the” include plural references, unless the content clearly dictates otherwise, and are used interchangeably with “at least one” and “one or more.” Thus, reference to “a polynucleotide” includes a plurality of polynucleotides or genes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The terms “gene” and “transgene” are used interchangeably. A “transgene” is a gene that has been transferred from one organism to another.

The term “subject” or “patient”, as used herein, refers to a domesticated animal, a farm animal, a primate, a mammal, for example, a human.

The phrase “substantially identical”, as used herein, refers to an amino acid sequence exhibiting high identity with a reference amino acid sequence (for example, at least 80%, at least 85%, at least 90%, at least 95%, at least 98%, or at least 99% identity) and retaining the biological activity of interest (the enzyme activity).

“Codon optimization” refers to the process of altering a naturally occurring polynucleotide sequence to enhance expression in the target organism, e.g., humans. In one embodiment of the subject application, the human MUT gene has been altered to replace codons that occur less frequently in human genes with those that occur more frequently and/or with codons that are frequently found in highly expressed human genes.

As used herein, “determining”, “determination”, “detecting”, or the like are used interchangeably herein and refer to the detecting or quantitation (measurement) of a molecule using any suitable method, including immunohistochemistry, fluorescence, chemiluminescence, radioactive labeling, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like. “Detecting” and its variations refer to the identification or observation of the presence of a molecule in a biological sample, and/or to the measurement of the molecule's value.

As used herein, a “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In certain embodiments, it may be preferable to include isotonic agents, for example, sugars, polyalcohols such as mannitol, sorbitol, or sodium chloride in the composition.

A “therapeutically effective amount” refers to an amount effective, at dosages and for periods of time necessary, to achieve the desired therapeutic result. A therapeutically effective amount of a vector comprising the synthetic polynucleotide of the invention may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the synthetic polynucleotide or a fragment thereof according to the invention calculated to produce the desired therapeutic effect in association with a pharmaceutical carrier.

### Products

Methylmalonic acidemia (MMA) is an autosomal recessive disorder caused by defects in the mitochondrial localized enzyme methylmalonyl-CoA mutase (MUT). The estimated incidence of MMA is 1 in 25,000-48,000. As used herein, “MUT” can refer to a human methylmalonyl coenzyme A mutase enzyme, and “Mut” can refer to a mouse methylmalonyl coenzyme A mutase enzyme, including variants thereof. MUT may also refer to methylmalonyl-CoA mutase from any species of mammal, including variants thereof. This protein catalyzes the isomerization of methylmalonyl-CoA to succinyl-CoA. This process requires 5′-deoxyadenosylcobalamin, a vitamin B12 derivative. Succinyl-CoA is a component of the citric acid cycle or tricarboxylic acid cycle (TCA). The gene or nucleotide or CDS or variants thereof encoding naturally occurring methylmalonyl coenzyme A mutase gene can be referred to variously as Mut (murine) or MUT (human, or synthetic). MUT/Mut and MUT/Mut can be used interchangeably herein with the nucleotide or protein referred to, either synthetic or WT, or murine, human or other species, or variants thereof, being apparent from the context in which the term appears.

Viral gene therapy has been used as treatment for MMA, using preclinical cellular and animal models to gather efficacy and safety data. A mouse model has been described wherein targeted deletion of methylmalonyl-CoA mutase (Mut) was used to create a mouse model of vitamin B12 non-responsive MMA that displays neonatal lethality. These (C57BL6x129SvEv) Mut^{-/-} mice faithfully replicate the phenotype of the most severely affected group of MMA patients who perish in the first 2 days of life. Although Mut^{-/-} mice display a phenotype that made many studies technically challenging, this model can be used to evaluate of interventions that have a strong effect on phenotype, such as systemic gene therapy, which can rescue the lethal neonatal phenotype.

Adeno-associated virus (AAV) gene therapy vectors have been developed of serotypes 2, 8 and 9 that express the murine or human MUT gene under the control of an enhanced, chicken beta-actin promoter (CBA) or the liver-specific, thyroid-binding globulin promoter (TBG) and have delivered them to Mut^{-/-} mice in the neonatal period. The results in the studies are striking: while the untreated Mut^{-/-} mice uniformly perish in early life, the treated Mut^{-/-} mice have near normal long-term survival, behave normally, display an ameliorated metabolic phenotype and demonstrate enzymatic activity as long as two years after treatment with an AAV8 or AAV9 vector. Surprisingly, the systemic delivery of an AAV9 vector also resulted in modest transduction of the kidney and long-term preservation of renal function in the treated mutants.

Because [(C57BL6x129SvEv)xFVB/N] Mut^{-/-} mice were produced in low numbers and rarely survived until weaning, an alternative approach to create animals that manifest inducible and/or intermediate phenotypes and display consistent, disease associated phenotypes has been required. We have therefore used transgenesis to model extra hepatic disease manifestations, such as chronic renal disease, metabolic strokes of the basal ganglia, and optic nerve atrophy.

A series of transgenic rescue constructs was engineered using well-established promoters and enhancers, and various transgenic lines have been generated. These new MMA mouse models can be used to model the hepato-renal disease of MMA by expression of the Mut enzyme in the muscle and another can be used to recapitulate the Mut− state though the ubiquitous expression of a partial deficiency mutation.

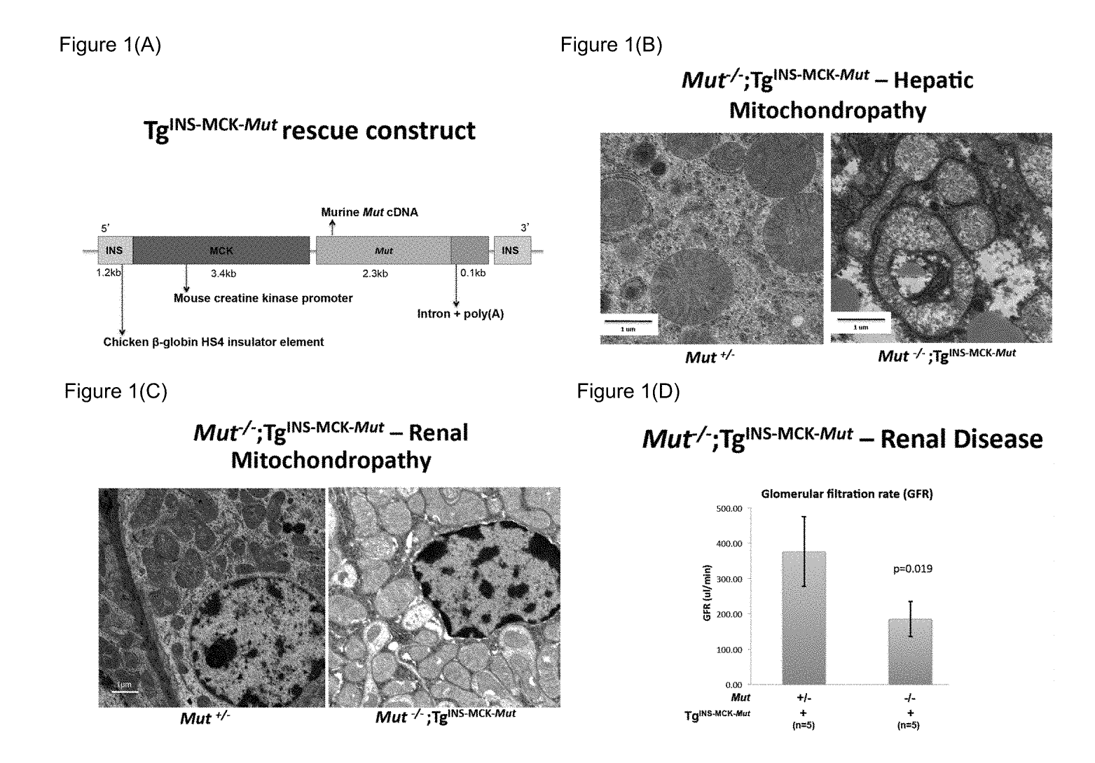

Mice that express the Mut enzyme only in the muscle, Mut^{-/-};Tg^{INS-MCK-Mut} mice, were generated using the murine creatine kinase (MCK) promoter to drive the expression of a Mut cassette that had been insulated with cHS4 barrier elements. These animals are viable but display massive elevations of serum metabolites, severe growth retardation, and precisely replicate the hepatorenal mitochondropathy and renal failure seen in patients (see, e.g., FIG. 1 (A)-1(D)). Mut^{-/-};Tg^{INS-MCK-Mut} mice are fragile and require a high carbohydrate, high fat diet as well as heating pads to survive, but despite these measures, only attain 40% of the bodyweight of age, sex and diet matched littermates. The phenotype can be corrected by systemic gene delivery with a single wild-type Mut allele, indicating that the disease manifestations are entirely attributable to the metabolic defect.

To complement the Mut tissue-specific expression models, mice with Mut−, or partial deficiency MMA, were also developed. Clinically, Mut− patients are less severely affected than those that are the Mut^{o} subtype yet they still can exhibit significant disease manifestations, including hyperammonemia, renal insufficiency, pancreatitis, a propensity to develop metabolic strokes and reduced survival. Mice that are Mut− are easier to breed and manipulate yet can be induced to develop severe symptoms by simple experimental means, such as the intake of a high protein diet or intraperitoneal propionic acid injections.

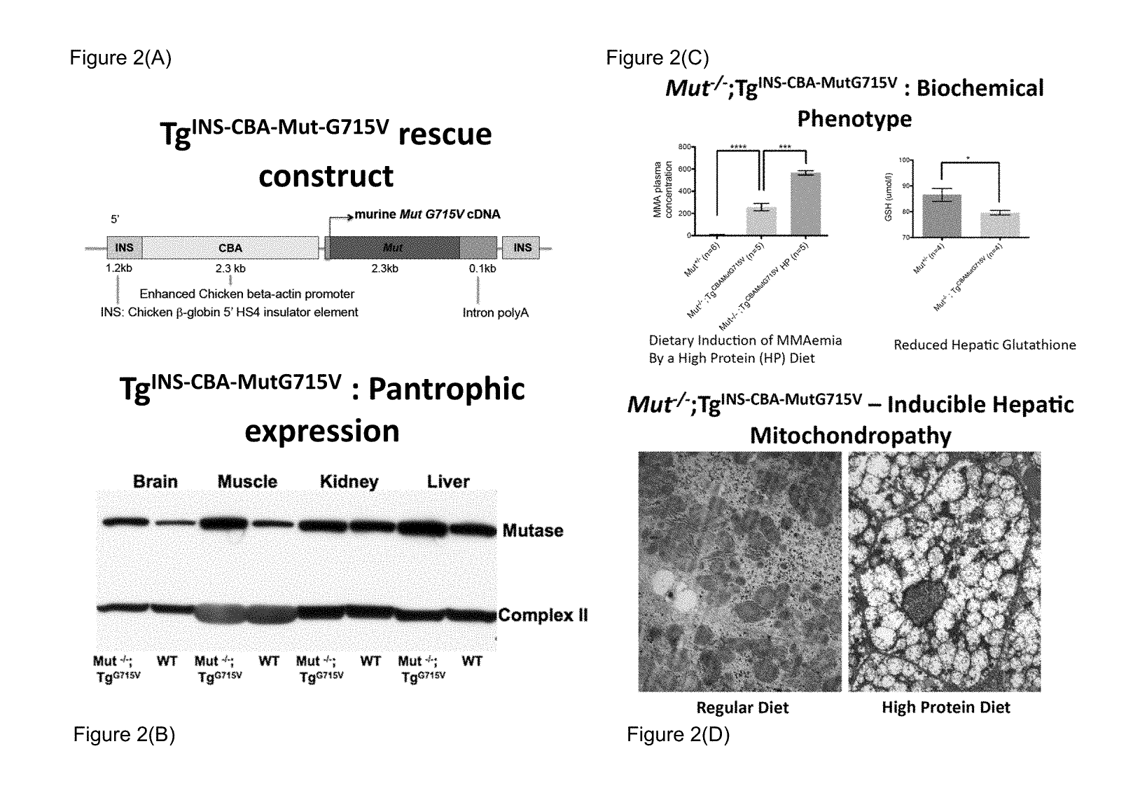

To create a mouse model of Mut− MMA, a homolog of a well-characterized human mutation, MUT p.G717V has been introduced into mice. This mutant protein cannot bind to the cofactor, 5′deoxyadenosylcobalamin, and therefore is inactive under physiological vitamin concentrations, recapitulating the common cofactor Kₘ class of MUT mutation. We used site-directed mutagenesis to generate the homologous mouse mutation, Mut p.G715V, and determined that the mouse p.G715V enzyme possesses the same defect in adenosylcobalamin binding as the human p G717V enzyme, with a measured Kₘ for adenosylcobalamin of 3.5×10⁻⁵ M, which is three orders of magnitude more than the wild type enzyme. Insulated transgenic expression constructs, with the murine mutant cDNA driven by the chicken actin promoter and flanked by insulators, have been used to derive transgenic lines. Mut^{-/-};Tg^{CBAMutG715V} mice constitutively express the mutant enzyme in all tissues examined. Lines expressing the wild-type murine cDNA in the same construct, (Mut^{-/-};Tg^{CBAMut}) were generated as a control for these studies and show complete biochemical correction at baseline and under high protein challenge. The Mut^{-/-};Tg^{CBAMutG715V} mice have methylmalonic acidemia/uria, allowing them to grow and develop normally on a high fat and carbohydrate diet. However, they develop massive methylmalonic acidemia, a hepatic mitochondropathy with decreased glutathione and weight loss when challenged with either high protein or isoleucine/valine enriched diets (see, e.g., FIG. 2(A)-(D)). The mouse models described above (muscle specific (Mut^{-/-};Tg^{INS-MCK-Mut}) and partial deficiency (Mut^{-/-};Tg^{CBAMutG715V})) more fully replicate MMA.

Both mouse models express the Mut enzyme either in the skeletal muscle or constitutively as a mutant cross-reactive material (CRM) positive allele. As many MMA patients are CRM positive or harbor missense mutations, gene therapy vectors that allow the detection of a functional Mut or MUT enzyme are needed for preclinical efficacy, expression and biodistribution studies. These mouse models are useful to determine the activity and biodistribution of synthetic MUT and Mut of the present invention introduced by gene therapy vector.

Accordingly, the subject invention generally relates to engineering of a novel gene therapy vector that expresses a biologically active murine methylmalonyl-CoA mutase (MUT or Mut) enzyme, internally tagged with an immunoaffinity and detection epitope, which is optionally flanked by linker sequences, which in some embodiments, have been designed and tested in mice. In one embodiment, the invention includes a synthetic methylmalonyl-CoA mutase (MUT) polypeptide comprising, in order from the N-terminus: a methylmalonyl-CoA mutase mitochondrial leader amino acid sequence, a tag amino acid sequence, and a methylmalonyl-CoA mutase mature amino acid sequence. The methylmalonyl-CoA mutase enzymes of the invention are thus internally tagged with an immunoaffinity and detection epitope. Optionally, the tag amino acid sequence is flanked by at least one linker amino acid sequence. In one embodiment, the synthetic enzymes of the invention contain a mitochondrial leader sequence fused to a tag epitope placed in a region of the murine, human or human codon-optimized methylmalonyl-CoA mutase enzyme that maintains mitochondrial localization and function and is coded for by nucleotides of the invention. The tag-methylmalonyl-CoA mutase fusion enzyme in one embodiment is found to have full biological activity in vivo, as well as therapeutic levels of expression, and the epitope tag allows for facile detection when co-expressed with an endogenous missense mutation of Mut. These genetically engineered, non-naturally occurring DNA sequences, and variants thereof, which encode murine or human Mut/MUT, can be used to express biologically functional methylmalonyl-CoA mutase and are useful to assay both activity and biodistribution after gene therapy in varied models of methylmalonic acidemia (MMA).

The nucleotides and polypeptides of the invention are also referred to variously herein as, for example, murine and human tag-Mut/MUT nucleotides, murine and human tag-MUT polypeptides or amino acids, and human tag synMUT1-4 (codon optimized human MUT), tag-MUT fusion enzyme, synthetic or engineered tag-MUT enzyme, tag-MUT protein, tag-Mut genes or transgenes, and the like. The “tag” may be optionally replaced by the specific tag, e.g., HA, V5, 3xFLAG and the like, as explained more fully herein.

In one embodiment, the present invention includes a MUT mitochondrial leader sequence in the N-terminal position of the fusion protein. Such methylmalonyl-CoA mutase mitochondrial leader sequences include human methylmalonyl-CoA mutase mitochondrial leader sequences and murine methylmalonyl-CoA mutase mitochondrial sequences. In one embodiment, the mitochondrial leader sequences include an amino acid sequence such as SEQ ID NO:1 (murine Mut mitochondrial sequence) and/or SEQ ID NO:2 (human MUT mitochondrial sequence), and/or an amino acid sequence having substantial identity thereto, e.g., at least about 95% identity thereto, and having substantially identical activity to the methylmalonyl-CoA mutase mitochondrial leader amino acid sequence.

In one embodiment, the present invention includes a methylmalonyl-CoA mutase mature amino acid sequence (functional enzyme) in the C-terminal position of the fusion protein. Such methylmalonyl-CoA mutase mature amino acid sequences include human methylmalonyl-CoA mutase mature amino acid sequences and murine methylmalonyl-CoA mutase mature amino acid sequences. In one embodiment, the methymalonyl-CoA mutase mature amino acid sequence may include SEQ ID NO:17 (murine methylmalonyl-CoA mutase mature) and/or SEQ ID NO:18 (human methylmalonyl-CoA mutase mature), and/or an amino acid sequence having substantial identity thereto, e.g., at least about 95% identity thereto, and having substantially identical activity to the methylmalonyl-CoA mutase mitochondrial leader amino acid sequence.

To create the synthetic methylmalonyl-CoA mutase polypeptides and nucleic acids of the invention, the inventors identified potential sites within the structure of methylmalonyl-CoA mutase that might tolerate the placement of an epitope tag. It was found that that the domain near the N-terminus of the mature enzyme was relatively flexible and solvent accessible when a V5 tag was engineered into this location. Because vertebrate methylmalonyl-CoA mutase enzymes have an obligate requirement for mitochondrial localization, the tag was incorporated behind the putative location of the mitochondrial leader sequence, which is cleaved by the mitochondrial protease when methylmalonyl-CoA mutase is imported into the mitochondrial inner space where it needs to localize for function. Appropriate flanking sequences were also chosen.

It was found that engineering the epitope tag into other positions within the enzyme produced inactive enzyme, indicating that this epitope tag was placed in or interfered with a critical enzymatic domain, such as the cobalamin binding pocket of the enzyme.

Therefore, the present invention includes synthetic, internally tagged MUT enzymes as disclosed in the present invention, which were developed and tested for efficacy in a mouse model of severe disease (Mut^{-/-};Tg^{INS-MCK-Mut}) and one that recapitulates the CRM positive state seen in many patients (Mut^{-/-};Tg^{CBAMutG715V}). In one embodiment of the present invention, the 5′ end of the Mut gene was replaced with an engineered nucleotide sequence that encodes the endogenous mitochondrial importation sequence, a mitochondrial protease cleavage site, and a V5 tag.

The V5 epitope tag, or V5 tag, GKPIPNPLLGLDST, is derived from the P/V proteins of paramyxovirus SV5. This V5 tag is also commonly used in such as mammalian and insect cell expression vectors and is understood by practitioners of the art. In some embodiments, other epitope tags which are known in the art may be used, such as, for example, without limitation, Myc-tag (EQKLISEEDL); HA-tag (Human influenza hemagglutinin YPYDVPDYA), FLAG (motif DYKXXD, commonly DYKDHDG-DYKDHDI-DYKDDDDK); His tag (e.g., poly(His)); TC tag (tetracysteine); VSVtag (Vesicular stomatitis virus (YTDIEMNRLGK)); Xpress tag (DLYDDDDK); Isopeptag (a peptide which binds covalently to pilin-C protein (TDKDMTITFTNKKDAE); SpyTag, a peptide which binds covalently to SpyCatcher protein (AHIVMVDAYKPTK); BCCP (Biotin Carboxyl Carrier Protein); GST (Glutathione-S-transferase-tag); thioredoxin tag; Fc-tag, derived from immunoglobulin Fc domain; and other peptide tag sequences as are known in the art, are used in place of, or in addition to, V5, for example, in the same location as the V5 tag.

Thus, in one embodiment, the present invention relates to a synthetic methylmalonyl-CoA mutase (MUT) polypeptide which is internally tagged with an epitope tag. This polypeptide includes an amino acid sequence tag which includes SEQ ID NO:8, SEQ ID NO:9, SEQ ID NO:10, SEQ ID NO:11, SEQ ID NO:12, SEQ ID NO:13, SEQ ID NO:14, SEQ ID NO:15, and/or SEQ ID NO:16.6, and/or an amino acid sequence having substantial identity thereto, e.g., at least about 95% identity thereto and retaining the function of an epitope tag.

In one embodiment, the synthetic polypeptide of the invention further includes linker sequences. These linker sequences, in one embodiment, can flank either side of the epitope tag. Appropriate flanking or linker sequences can be chosen by one of skill in the art. These flanking sequences may also be termed linker sequences. Therefore methylmalonyl-CoA mutase polypeptides of the invention may further comprise a linker between the epitope tag and the remainder of the polypeptide. The linker can be a sequence of about 1 to about 15 amino acids, between about 1 and about 12 amino acids, between about 1 and about 10 amino acids, between about 1 and about 8 amino acids, between about 1 and about 6 amino acids in length. The linker, when longer than one amino acid, may optionally include at least two different amino acids. If desired, a linker may be present between the mitochondrial leader sequence and the tag, and between the tag and the mature sequence. The linker increases the distance between the mitochondrial leader sequence, the mature protein, and the tag. The linker also allows for correct protein folding. In some embodiments, the linker contains at least one amino acid selected from the group consisting of serine (S), alanine (A), lysine (K), tyrosine (Y), threonine (T), phenylalanine (F), and glycine (G). Five non-limiting examples of useful linker sequences are MSYY (SEQ ID NO: 3), SKEFGT (SEQ ID NO: 4), GG (SEQ ID NO: 5), GGSS (SEQ ID NO: 6), and/or G (SEQ ID NO: 7). Suitable peptide linker sequences include a number of different sequences not named herein, which allow for enzyme activity and proper folding of both enzyme and tag, and may be chosen using algorithms, for example. In one embodiment, the linker is a non-cleavable linker.

Specific embodiments of linker-tag-linker include, for example, an amino acid sequence comprising SEQ ID NO:36 (GGSSYPYDVPDYAGG), SEQ ID NO:37 (GGSSDYKDDDDKGDYKDDDDKGDYKDDDDKGG), and/or SEQ ID NO:38 (MSYYGKPIPN PLLGLDSTSKEFGT). These tag-linker-tag sequences may be inserted into a MUT protein between the mitochondrial leader sequence and the mature sequence.

Therefore, in an embodiment, the present invention includes the fully assembled fusion proteins comprising a methylmalonyl-CoA mutase mitochondrial leader sequence, a linker, a tag, a linker, and a MUT/Mut or variant thereof, enzyme sequence. Such fusion proteins include, for example, SEQ ID NO:19 (murine V5-Mut), SEQ ID NO:20 (murine V5-Mut mature sequence), SEQ ID NO:21 (human V5-MUT), SEQ ID NO:22 (human V5-MUT mature sequence), SEQ ID NO:23 (human HA-MUT sequence derived from, e.g., expression of HA-SynMUT4), SEQ ID NO:24 (human HA-MUT mature sequence derived from, e.g., expression of HA-SynMUT4), SEQ ID NO:25 (human 3xFLAG-MUT sequence derived from, e.g., expression of 3xFLAG-SynMUT4), SEQ ID NO:26 (human 3xFLAG-MUT mature sequence derived from, e.g., expression of 3xFLAG-SynMUT4), and/or an amino acid sequence having substantial identity thereto, e.g., at least about 95% identity thereto, and having substantially identical activity to the methylmalonyl-CoA mutase mature amino acid sequence.

The present invention also relates to a synthetic methylmalonyl-CoA mutase (Mut or MUT) nucleic acid sequence or polynucleotide which encodes a synthetic methylmalonyl-CoA mutase (Mut or MUT) amino acid or polypeptide sequences as disclosed herein. In specific embodiments, synthetic polynucleotides which encode polypeptides of the present invention include nucleic acid sequences such as SEQ ID NO:27 (V5-synMut1), SEQ ID NO:28 (murine V5-Mut), SEQ ID NO:29 (HA-synMUT4), SEQ ID NO:30 (3xFLAG-synMUT4), and/or a nucleic acid sequence having substantial identity to, e.g., at least about 95% identity thereto, wherein the synthetic polynucleotide encodes for a polypeptide that has substantially identical activity to WT methylmalonyl-CoA mutase.

In one embodiment, the present invention includes polynucleotides comprising a codon optimized MUT or Mut allele. An example of codon optimized Mut or MUT alleles suitable for the present invention are disclosed in, for example, International Application PCT/US2014/028045, filed Mar. 14, 2014, U.S. Ser. No. 61/792,081, filed Mar. 15, 2015, and U.S. Ser. No. 15/070,787, filed Mar. 15, 2016, all of which are incorporated herein by reference in their entireties, and for each of the codon-optimized sequences that they disclose, and wherein their disclosed codon-optimized MUT alleles are specifically incorporated by reference herein. In this reference, disclosed are highly active and synthetic MUT alleles, called synMUT1-4, which provide for increased expression of methylmalonyl-CoA mutase. In one embodiment, the subject synthetic polynucleotide includes a polynucleotide which encodes a polypeptide with 100% identity to the naturally occurring human methylmalonyl-CoA mutase protein, alternatively including naturally occurring alleles, and may include, without limitation, a polynucleotide such as, for example, SEQ ID NO:31 (CDS, human MUT), SEQ ID NO:32 (CDS, synMUT1), SEQ ID NO:33 (CDS, synMUT2), SEQ ID NO:34 (CDS, synMUT3), SEQ ID NO:35 (CDS, synMUT4), and/or a polynucleotide sequence having substantial identity to, e.g., at least about 95% identity thereto, wherein the synthetic polynucleotide encodes for a polypeptide that has substantially identical activity to WT MUT. The polynucleotide comprising a polynucleotide encoding MUT may further comprise a polynucleotide encoding a polypeptide comprising a tag and/or at least one linker sequence as disclosed herein. In one embodiment polypeptide comprising a tag and/or at least one linker sequence is inserted between the sequences encoding the mitochondrial leader sequence and the mature protein, e.g., between nucleotides 96 and 97 of the named Mut or MUT polynucleotide sequences.

The tag and/or linker may comprise a polynucleotide encoding a polynucleotide tag as described herein. In one embodiment the polypeptide tag can include SEQ ID NO:8, SEQ ID NO:9, SEQ ID NO:10, SEQ ID NO:11, SEQ ID NO:12, SEQ ID NO:13, SEQ ID NO:14, SEQ ID NO:15, and/or SEQ ID NO:16. The optional linker may comprise at least one polynucleotide encoding a polypeptide linker as described herein. In one embodiment, the polypeptide includes MSYY (SEQ ID NO: 3), SKEFGT (SEQ ID NO: 4), GG (SEQ ID NO: 5), GGSS (SEQ ID NO: 6), and/or G (SEQ ID NO: 7); or the tag/linker may include a polynucleotide selected from the group consisting of SEQ ID NO:36, SEQ ID NO:37, and/or SEQ ID NO:38.

In another aspect, the invention is directed to an expression vector comprising the herein-described synthetic polynucleotide. In another embodiment of a vector according to the invention, the synthetic polynucleotide is operably linked to an expression control sequence.

In one aspect, the invention is directed to a transgenic cell or an animal whose genome comprises the synthetic polynucleotide described herein. In another aspect, the invention is directed to a method for producing such a transgenic cell or animal, comprising: providing an exogenous expression vector comprising a polynucleotide comprising a promoter operably linked to the synthetic polynucleotide described herein; introducing the vector into a cell or a fertilized oocyte; and culturing the cell or transplanting the oocyte into a female animal. Humans are excluded from the definition of animal in this embodiment.

Methods for producing transgenic cells or animals are known in the art and include, without limitation, transforming cells or embryonic stem cells in tissue culture, injecting the transgene into the pronucleus of a fertilized animal egg (DNA microinjection), genetic/genome engineering, viral delivery (for example, retrovirus-mediated gene transfer), or culturing cells.

Transgenic animals according to the invention include, without limitation, rodent (mouse, rat, squirrel, guinea pig, hamster, beaver, porcupine), frog, ferret, rabbit, chicken, pig, sheep, goat, cow primate, and the like.

In one embodiment, the polypeptides of the present invention have at least 70%, at least about 75%, at least about 80%, at least about 85%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, amino acid identity to the sequences disclosed herein. In another embodiment, the polynucleotides of the present invention have at least 70%, at least about 75%, at least about 80%, at least about 85%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, nucleotide identity to the sequences disclosed herein. Percent (%) amino acid/nucleotide sequence identity herein is defined as the percentage of amino acid residues/nucleotides in a candidate sequence that are identical with the amino acid residues/nucleotides in a selected sequence, after aligning the sequences and introducing gaps, if necessary, to achieve the maximum percent sequence identity, and not considering any conservative substitutions as part of the sequence identity. Alignment for purposes of determining percent amino acid/nucleotide sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

The polynucleotides that encode the nucleotides of the present invention may contain further nucleotide alterations, including substitutions and/or insertions and/or deletions in any other region of the Mut or MUT nucleotide, including the 5′- and 3′-terminal coding regions. Preferably, these substitutions will be “conservative” substitutions and do not alter the amino acid residues of the resultant polypeptides. In some embodiments, an amino acid residue may be altered, but the change is a change to another amino acid that is similar to the one replaced and the structure and/or function of the resultant polypeptides will remain.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

In one embodiment, a polypeptide according to the invention retains at least 50%, at least 55%, at least 60%, at least 65%, at least 70%, at least 75%, at least 80%, at least 85%, at least 90% of the naturally occurring human or murine MUT protein function, i.e., the capacity to catalyze the conversion of L-methylmalonyl-CoA to succinyl-CoA and/or the capacity to be localized to the mitochondria upon expression. In another embodiment, a polypeptide of the present invention retains at least 95% of the naturally occurring human MUT protein function. In yet another embodiment, a polypeptide of the invention has substantially identical activity to WT MUT. Alternatively, the polypeptide can have substantially identical expression to, or greater expression than, to a WT MUT. This protein function and/or expression level can be measured, for example, via the efficacy to ameliorate the phenotype in Mut knock-out mice (Chandler, et al. 2010 Mol Ther 18:11-6), the lowering of circulating metabolites including methylmalonic acid in a disease model of MMA (Chandler, et al. 2010 Mol Ther 18:11-6; Carrillo-Carrasco, et al. 2010 Hu Gene Ther 21:1147-54; Senac, et al. 2012 Gene Ther 19:385-91), the measurement of whole body (Chandler, et al. 2010 Mol Ther 18:11-6; Senac, et al. 2012 Gene Ther 19:385-91) or hepatic 1-C-¹³propionate oxidative capacity (Carrillo-Carrasco, et al. 2010 Hu Gene Ther 21:1147-54), or the correction of macromolecular 1-C-¹⁴propionate incorporation in cell culture (Chandler, et al. 2007 BMC Med Genet 8:64).

The synthetic polynucleotide can be composed of DNA and/or RNA or a modified nucleic acid, such as a peptide nucleic acid, and could be conjugated for improved biological properties.

### Therapy

In one embodiment, gene delivery, such as AAV gene delivery, of the nucleotides of the invention is useful as a treatment for patients with methylmalonic acidemia, propionic acidemia as well as other organic acidemias, grave inborn errors of metabolism that, as a group, currently lack definitive therapy. The cumulative clinical observations that MMA liver transplant and liver/kidney recipients are effectively cured from the propensity to suffer lethal metabolic decompensations after transplantation, combined with mouse model gene therapy data demonstrating that low levels of persistent correction mediated by AAV gene therapy provide lifelong stability in a highly accurate disease model, provide a practical and theoretical approach for using gene therapy to treat patients with MMA.

In another aspect, the invention is directed to the preclinical amelioration or rescue from the disease state, for example, methylmalonic acidemia, that the afflicted subject exhibits. This may include symptoms, such as lethargy, lethality, metabolic acidosis, and biochemical perturbations, such as increased levels of methylmalonic acid in blood, urine, and body fluids.

In another aspect, the invention comprises a method of treating a disease or condition mediated by methylmalonyl-CoA mutase in a subject or patient. The disease or condition can, in one embodiment, be methylmalonic acidemia (MMA). This method comprises administering to a subject in need thereof a therapeutic amount of a nucleotide and/or a polypeptide of the present invention. Administration may be performed by methods known in the art, such as enzyme therapy, gene therapy or gene editing which result in expression of polypeptides of the invention, as well as by directly administering polypeptides.

In one embodiment, the expressed methylmalonyl-CoA mutase enzymes of the invention are processed after transcription, translation, and translocation into the mitochondrial inner space. During this importation and maturation process, for example amino acids 1-30 are removed to produce the mature methylmalonyl-CoA mutase, comprised of residues comprised of residues 31-748 or amino acids 1-32 are removed to produce the mature methylmalonyl-CoA mutase peptide, comprised of residues 33-750. Thus, in another embodiment, the invention might include the mature portion of the processed MUT or Mut of the invention located inside the mitochondrial matrix, attached to a carrier that recognizes the V5 or other tag, conjugated to a charged or lipophilic small molecule to direct toward the mitochondria; conjugated or covalently modified to a peptide that targets the mitochondrial matrix; or encapsulated to deliver this fragment of methylmalonyl-CoA mutase to a subcellular organelle, cell type or tissue.

Enzyme replacement therapy consists of administration of the functional enzyme (methylmalonyl-CoA mutase) to a subject in a manner so that the enzyme administered will catalyze the reactions in the body that the subject's own defective or deleted enzyme cannot. In enzyme therapy, the defective enzyme can be replaced in vivo or repaired in vitro using the synthetic polynucleotide according to the invention, using therapeutically effective amounts of the polypeptides and/or polynucleotides of the invention. The functional enzyme molecule can be isolated or produced in vitro, for example. Methods for producing recombinant enzymes in vitro are known in the art. In vitro enzyme expression systems include, without limitation, cell-based systems (bacterial (for example, Escherichia coli, Corynebacterium, Pseudomonas fluorescens), yeast (for example, Saccharomyces cerevisiae, Pichia Pastoris), insect cell (for example, Baculovirus-infected insect cells, non-lytic insect cell expression), and eukaryotic systems (for example, Leishmania)) and cell-free systems (using purified RNA polymerase, ribosomes, tRNA, ribonucleotides). Viral in vitro expression systems are likewise known in the art.

Gene therapy can involve in vivo gene therapy (direct introduction of the genetic material into the cell or body) or ex vivo gene transfer into a subject or patient, of the nucleotides of the present invention, which usually involves genetically altering cells prior to administration, resulting in therapeutically effective amounts of the nucleotides/polypeptides of the present invention in the subject/patient. This might include the administration of the sequences of this invention as a peptide nucleic acid, mRNA, modified mRNA, or other nucleic acid, either directly or through a nanoparticle, such as a lipid or polymer nanoparticle.

In another embodiment, genome editing, or genome editing with engineered nucleases (GEEN) may be performed with the nucleotides of the present invention allowing DNA to be inserted, replaced, or removed from a genome using artificially engineered nucleases. Any known engineered nuclease may be used such as Zinc finger nucleases (ZFNs), Transcription Activator-Like Effector Nucleases (TALENs), the CRISPR/Cas system, and engineered meganuclease re-engineered homing endonucleases. Alternately, the nucleotides of the present invention, in combination with a CASP/CRISPR, ZFN, or TALEN can be used to engineer correction at the locus in a patient's cell either in vivo or ex vivo, then, in one embodiment, use that corrected cell, such as a fibroblast or lymphoblast, to create an iPS or other stem cell for use in cellular therapy.

### Biodistribution

In one embodiment, the present invention includes a method for detecting the extent of expression of a nucleotide of the present invention in a subject or patient after introduction into the subject or patient, comprising detecting the expression of the tag. The nucleotide of the present invention may be introduced to the patient via gene therapy, gene editing, or by enzyme replacement therapy as disclosed herein. In one embodiment, the tag is a V5, HA tag or FLAG tag. Methods to detect the presence of a tag are disclosed herein, including immunohistochemistry, fluorescence, chemiluminescence, radioactive labeling, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like, of a patient or subject's tissue. “Detecting” and its variations refer to the identification or observation of the presence of a molecule in a biological sample, and/or to the measurement of the molecule's value.

### Administration/Delivery and Dosage Forms

Routes of delivery of a synthetic methylmalonyl-CoA mutase (MUT) polynucleotide according to the invention may include, without limitation, injection (systemic or at target site), for example, intradermal, subcutaneous, intravenous, intraperitoneal, intraocular, subretinal, renal artery, hepatic vein, intramuscular injection; physical, including ultrasound(-mediated transfection), electric field-induced molecular vibration, electroporation, transfection using laser irradiation, photochemical transfection, gene gun (particle bombardment); parenteral and oral (including inhalation aerosols and the like). Related methods include using genetically modified cells, antisense therapy, and RNA interference.

Vehicles for delivery of a synthetic methylmalonyl-CoA mutase polynucleotide according to the invention may include, without limitation, viral vectors (for example, AAV, adenovirus, baculovirus, retrovirus, lentivirus, foamy virus, herpes virus, Moloney murine leukemia virus, Vaccinia virus, and hepatitis virus) and non-viral vectors (for example, naked DNA, mini-circles, liposomes, ligand-polylysine-DNA complexes, nanoparticles, cationic polymers, including polycationic polymers such as dendrimers, synthetic peptide complexes, artificial chromosomes, and polydispersed polymers, mRNA, or base-modified mRNA). Thus, dosage forms contemplated include injectables, aerosolized particles, capsules, and other oral dosage forms.

In certain embodiments, the vector used for gene therapy comprises an expression cassette. The expression cassette may, for example, consist of a promoter, the synthetic polynucleotide, and a polyadenylation signal. Viral promoters include, for example, the ubiquitous cytomegalovirus immediate early (CMV-IE) promoter, the chicken beta-actin (CBA) promoter, the simian virus 40 (SV40) promoter, the Rous sarcoma virus long terminal repeat (RSV-LTR) promoter, the Moloney murine leukemia virus (MoMLV) LTR promoter, and other retroviral LTR promoters. The promoters may vary with the type of viral vector used and are well-known in the art.

In one specific embodiment, a synthetic methylmalonyl-CoA mutase polynucleotide according to the present invention could be placed under the transcriptional control of a ubiquitous or tissue-specific promoter, with a 5′ intron, polyadenylation signal, and mRNA stability element, such as the woodchuck post-transcriptional regulatory element. The use of a tissue-specific promoter can restrict unwanted transgene expression, as well as facilitate persistent transgene expression. The therapeutic transgene could then be delivered as coated or naked DNA into the systemic circulation, portal vein, or directly injected into a tissue or organ, such as the liver or kidney. In addition to the liver or kidney, the brain, pancreas, eye, heart, lungs, bone marrow, and muscle may constitute targets for therapy. Other tissues or organs may be additionally contemplated as targets for therapy.

In another embodiment, synthetic methylmalonyl-CoA mutase polynucleotide according to the present invention could be packaged into a viral vector, such as an adenoviral vector, retroviral vector, lentiviral vector, or adeno-associated viral vector, and delivered by various means into the systemic circulation, portal vein, or directly injected into a tissue or organ, such as the liver or kidney. In addition to the liver or kidney, the brain, pancreas, eye, heart, lungs, bone marrow, and muscle may constitute targets for therapy. Other tissues or organs may be additionally contemplated as targets for therapy.

Tissue-specific promoters include, without limitation, Apo A-I, ApoE, hAAT, transthyretin, liver-enriched activator, albumin, PEPCK, and RNAP_{II} promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, and myelin basic protein promoters (neuronal), and ankyrin, α-spectrin, globin, HLA-DRα, CD4, glucose 6-phosphatase, and dectin-2 promoters (erythroid).

Regulable promoters (for example, ligand-inducible or stimulus-inducible promoters) are also contemplated for expression constructs according to the invention.

In yet another embodiment, a synthetic methylmalonyl-CoA mutase polynucleotide according to the present invention could be used in ex vivo applications via packaging into a retro or lentiviral vector to create an integrating vector that could be used to permanently correct any cell type from a patient with Mut/MUT deficiency. The so-transduced and corrected cells could then be used as a cellular therapy. Examples might include CD34+ stem cells, primary hepatocytes, or fibroblasts derived from patients with Mut/MUT deficiency. Fibroblasts could be reprogrammed to other cell types using iPS methods well known to practitioners of the art. In yet another embodiment, a synthetic V5-methylmalonyl-CoA mutase polynucleotide or polypeptide according to the present invention could be recombined using genomic engineering techniques that are well known to practitioners of the art, such as ZFNs and TALENS, into the MUT locus, a genomic safe harbor site, such as AAVS1, or into another advantageous location, such as into rDNA, the albumin locus, GAPDH, or a suitable expressed pseudogene.

A composition (pharmaceutical composition) for treating an individual by gene therapy may comprise a therapeutically effective amount of a vector comprising a transgene or viral particle produced or obtained from the same, wherein the transgene or viral particle comprise the synthetic V5-methylmalonyl-CoA mutase, HA-methylmalonyl-CoA mutase, or 3XFLAG-methylmalonyl-CoA mutase polynucleotides according to the present invention. The pharmaceutical composition may be for human or animal usage. Typically, a physician will determine the actual dosage which will be most suitable for an individual subject, and it will vary with the age, weight, and response of the particular individual.

The composition may, in specific embodiments, comprise a pharmaceutically acceptable carrier, diluent, excipient, or adjuvant. Such materials should be non-toxic and should not interfere with the efficacy of the transgene. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences [Mack Pub. Co., 18th Edition, Easton, Pa. (1990)]. The choice of pharmaceutical carrier, excipient, or diluent can be selected with regard to the intended route of administration and standard pharmaceutical practice. The pharmaceutical compositions may comprise as, or in addition to, the carrier, excipient, or diluent any suitable binder(s), lubricant(s), suspending agent(s), coating agent(s), solubilizing agent(s), and other carrier agents that may aid or increase the viral entry into the target site (such as for example a lipid delivery system). For oral administration, excipients such as starch or lactose may be used. Flavoring or coloring agents may be included, as well. For parenteral administration, a sterile aqueous solution may be used, optionally containing other substances, such as salts or monosaccharides to make the solution isotonic with blood.

A composition according to the invention may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, modulators, or drugs (e.g., antibiotics).

The composition may be in a variety of forms. These include, for example, liquid, semi-solid and solid dosage forms, such as liquid solutions (e.g., injectable and infusible solutions), dispersions or suspensions, tablets, pills, powders, liposomes and suppositories. Additional dosage forms contemplated include: in the form of a suppository or pessary; in the form of a lotion, solution, cream, ointment or dusting powder; by use of a skin patch; in capsules or ovules; in the form of elixirs, solutions, or suspensions; in the form of tablets or lozenges.

Further details of the present invention will be apparent from the following non-limiting Examples.

### EXAMPLES

### Example 1

Gene therapy in methylmalonyl-CoA mutase Mut^{-/-};Tg^{INS-MCK-Mut} Mice. Mut^{-/-};Tg^{INS-MCK-Mut} mice express wild type Mut in the skeletal muscle under the control of the murine creatine kinase promoter and display severe biochemical perturbations (FIG. 1(A)-1(D)). The targeted Mut allele harbors a deletion of exon 3 in the Mut gene. This exon encodes the putative substrate-binding pocket in the MUT enzyme. The Mut allele does not produce mature RNA, protein, or enzymatic activity. Mut^{-/-};Tg^{INS-MCK-Mut} mice exhibit severe symptoms of hepatorenal disease and have massive metabolic elevations of methylmalonic acid in the blood and body fluids. These animals are also growth retarded like MMA patients. In the instant example, the Mut^{-/-};Tg^{INS-MCK-Mut} mouse is also referred to as a mouse with MMA.

A synthetic V5-murine methylmalonyl-CoA mutase gene (V5Mut) (SEQ ID NO:1) was engineered to incorporate an internal V5 tag that was predicted to not impair transport, processing or function of MUT and then synthesized. The V5Mut gene was then cloned using restriction endonuclease excision and DNA ligation. Cloning methods are well understood by practitioners of the art (Sambrook, Fritsch, Maniatis. Molecular Cloning: A Laboratory Manual).

The recombinant adeno-associated viral vector designed to express V5Mut in the liver and other tissues of the MMA mouse was prepared using restriction endonuclease excision and DNA ligation. The AAV2/9-CBA-RBG vector contains transcriptional control elements from the chicken β-actin promoter (Chandler, et al. 2010 Mol Ther 18:11-6), cloning sites for the insertion of a complementary DNA, and the rabbit β-globin polyadenylation (RBG) signal. Terminal repeats from AAV serotype 2 flank the expression cassette. The murine V5Mut gene was cloned into AAV2-CBA-RBG and packaged into rAAV9 as previously described (Senac, et al. 2012), purified by cesium chloride centrifugation, and titered by qPCR to make the AAV9-CBA-V5Mut RBG vector (SEQ ID NO:8) using methods previously described (Chandler, et al. 2010 Mol Ther 18:11-6; Carrillo-Carrasco, et al. 2010 Hu Gene Ther 21:1147-54). Animal studies were reviewed and approved by the National Human Genome Research Institute Animal User Committee. Retroorbital injections were performed on anesthetized Mut^{-/-};Tg^{INS-MCK-Mut} mice at weaning. Viral particles were diluted to a total volume of 50 microliters with phosphate-buffered saline immediately before injection and delivered as described (Senac et al).

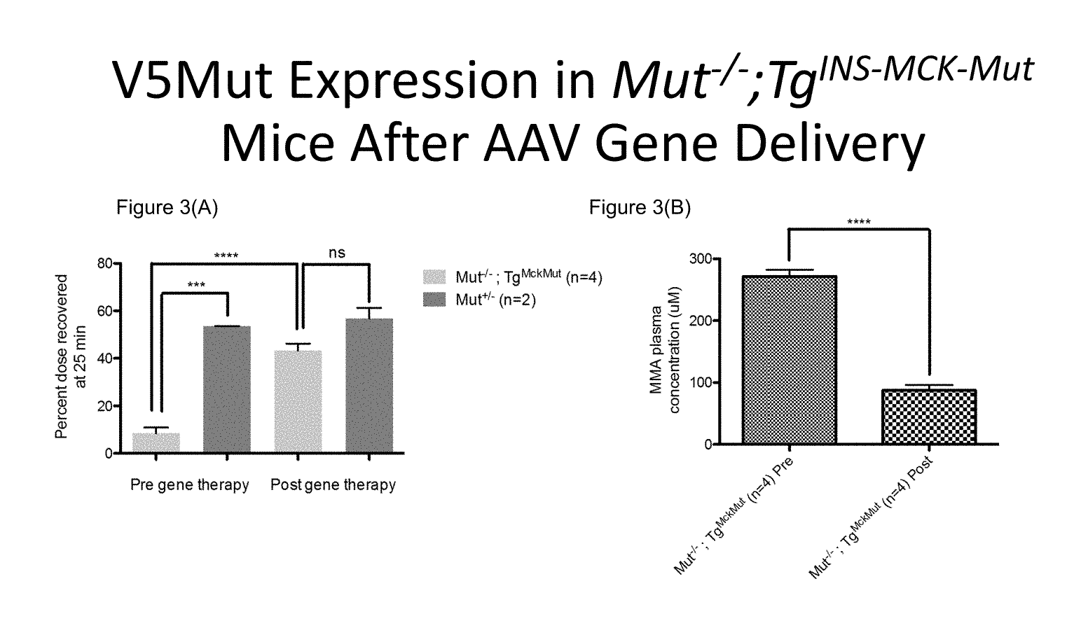

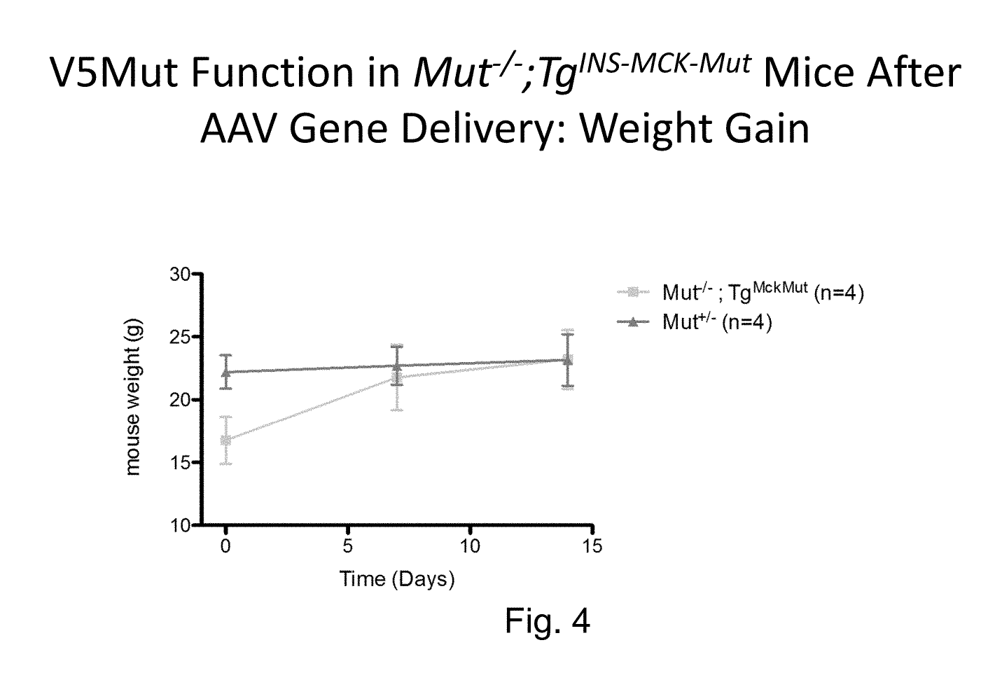

Treatment of Mut^{-/-};Tg^{INS-MCK-Mut} mice with V5Mut polynucleotide delivered using an AAV (adeno-associated virus) restored the whole body oxidative capacity to metabolize 1-C-¹³ labeled propionic acid, which is a direct precursor of methymalonic acid and unmetabolizeable without the action of MUT in the liver (FIG. 3(A)); lowered the levels of plasma methylmalonic acid in the blood (FIG. 3(B)); and improved growth to that seen in wild type mice (FIG. 4). These data establish the pre-clinical efficacy of V5Mut as a treatment for MMA in vivo, which would be expected to translate to other animal models as well as humans.

### Example 2

Gene therapy in methylmalonyl-CoA mutase Mut^{-/-};Tg^{CBAMutG715V} Mice. A homolog of a well-characterized human mutation, MUT p.G717V, was introduced into mice to make a model of partial deficiency MMA. The transgene resides in trans to the Mut locus. The targeted Mut allele harbors a deletion of exon 3 in the Mut gene. This exon encodes the putative substrate-binding pocket in the Mut enzyme. The Mut allele does not produce mature RNA, protein, or enzymatic activity. The Mut^{-/-};Tg^{CBAMutG715V} mice have methylmalonic acidemia/uria, allowing them to grow and develop normally on a high fat and carbohydrate diet. However, they develop massive methylmalonic acidemia, a hepatic mitochondropathy with decreased glutathione and weight loss when challenged with either high protein or isoleucine/valine enriched diets (see FIG. 2A-2D). In the instant example, the Mut^{-/-};Tg^{CBAMutG715V} mouse is also referred to as a mouse with MMA.

A synthetic V5-murine methylmalonyl-CoA mutase gene (V5Mut) was engineered to incorporate an internal V5 tag was then synthesized. The V5Mut gene was then cloned using restriction endonuclease excision and DNA ligation. Cloning methods are well understood by practitioners of the art (Sambrook, Fritsch, Maniatis. Molecular Cloning: A Laboratory Manual).

A recombinant recombinant adeno-associated viral vector designed to express V5Mut in the liver and other tissues of the MMA mouse was prepared using restriction endonuclease excision and DNA ligation. The AAV2/9-CBA-RBG vector contains transcriptional control elements from the chicken β-actin promoter (Chandler, et al. 2010 Mol Ther 18:11-6), cloning sites for the insertion of a complementary DNA, and the rabbit β-globin polyadenylation (RBG) signal. Terminal repeats from AAV serotype 2 flank the expression cassette. The murine V5Mut gene was cloned into AAV2-CBA-RBG and packaged into rAAV9 as previously described (Senac, et al. 2012), purified by cesium chloride centrifugation, and titered by qPCR to make the AAV9-CBA-V5Mut RBG vector using methods previously described (Chandler, et al. 2010 Mol Ther 18:11-6; Carrillo-Carrasco, et al. 2010 Hu Gene Ther 21:1147-54). Animal studies were reviewed and approved by the National Human Genome Research Institute Animal User Committee. Retroorbital injections were performed on anesthetized Mut^{-/-};Tg^{CBAMutG715V} mice at weaning. Viral particles were diluted to a total volume of 50 microliters with phosphate-buffered saline immediately before injection and delivered as described (Senac et al 2012). The organs were harvested in the mice two weeks after injection and a single mouse liver was studied for protein expression.

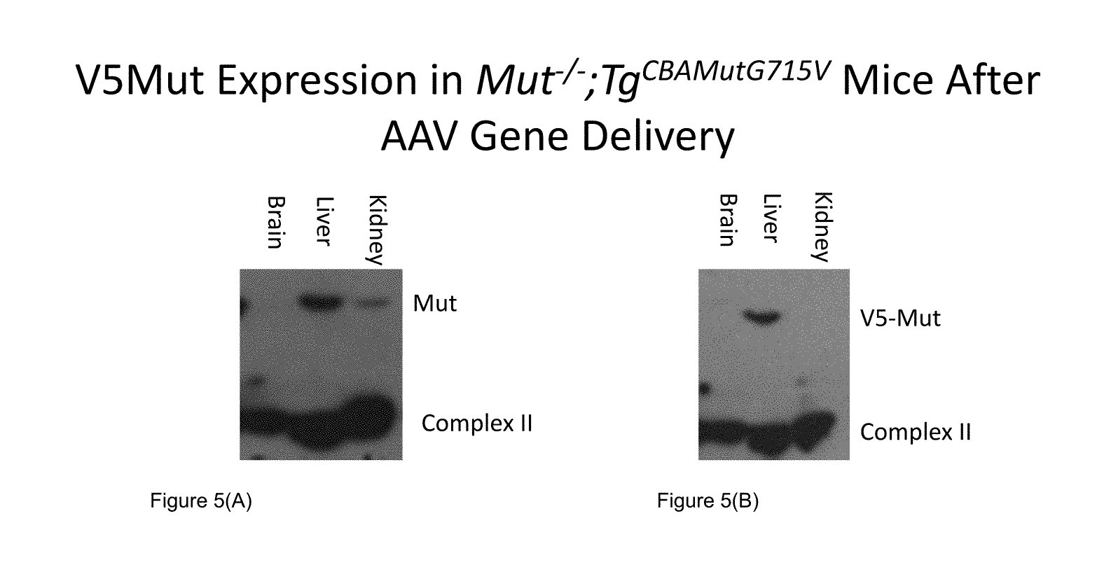

A liver extract was prepared and 25 milligrams used to perform a Western blot, probing with either anti-MUT or anti-V5 antibodies. Complex II was used as a loading control. Treatment of Mut^{-/-};Tg^{CBAMutG715V} mice with V5Mut polynucleotide delivered using an AAV (adeno-associated virus) improved Mut expression in the liver (FIG. 5(A)) and allowed for detection of the viral transgene via the V5 tag in the liver (FIG. 5(B)). These data establish the efficacy of V5Mut as expressed from the gene therapy vector on the background of a CRM positive allele—pG715V Mut— and demonstrate that the V5 tag is detectable by Western analysis.

### Example 3

A synthetic HA-synMUT4 methylmalonyl-CoA mutase gene (HA-synMUT4) (SEQ ID NO:29) was engineered to incorporate an internal HA tag that was predicted to not impair transport, processing or function of MUT and then synthesized. The HA-synMUT4 gene was then cloned using restriction endonuclease excision and DNA ligation. A synthetic 3XFLAG-synMUT4 methylmalonyl-CoA mutase gene (3XFLAG-synMUT4) (SEQ ID NO:30) was engineered to incorporate an internal 3XFLAG tag that was predicted to not impair transport, processing or function of MUT and then synthesized. The 3XFLAG-synMUT4 gene was then cloned using restriction endonuclease excision and DNA ligation. Cloning methods are well understood by practitioners of the art (Sambrook, Fritsch, Maniatis. Molecular Cloning: A Laboratory Manual).

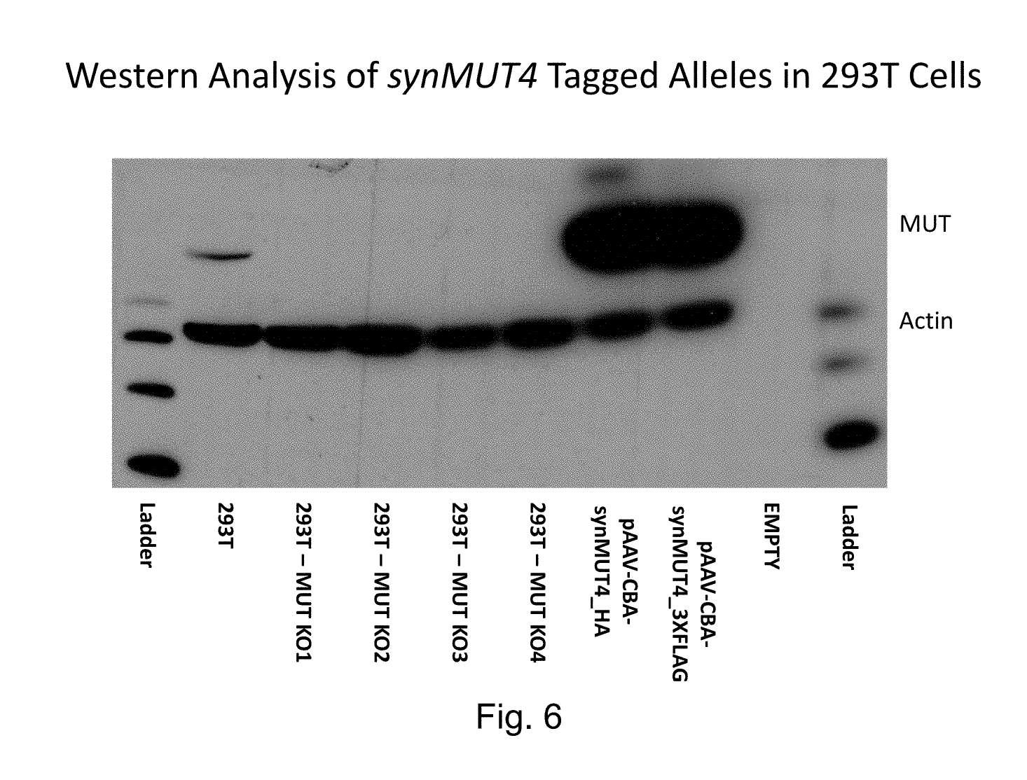

Each MUT allele described above was cloned into an AAV vector under the control of the enhanced chicken beta actin (CBA) promoter. 293T cells were transfected with 5 μg of the DNA vector and expression of the varied MUT transgenes was studied by Western Blotting (FIG. 6). Actin served as a control. 20 μg of cell lysate was subjected to Western analysis and probed with anti-MUT antibody or anti-actin antibody. The intensity of the endogenous MUT band is noted in lane 2. 293T clonal cell lines engineered to harbor a MUT knock out allele are in the next 4 lanes, followed by the 293T cells transfected with AAV constructs expressing either HA-synMUT4 or 3xFLAG-synMUT4. As can be seen, HA-synMUT4 and 3xFLAG-synMUT4 produce abundant immunoreactive MUT, indicating that the tag has not interfered with stability or localization, both of which can induce degradation of MUT.

The cumulative results with both mouse models show in vivo efficacy, as proven by the phenotypic and metabolic correction of a severely affected MMA mouse in the case of Mut^{-/-}; Tg^{MckMut} and further functionality of the V5 tag to monitor expression of the VSMut enzyme at the protein level in the instance of the Mut^{-/-};Tg^{CBAMutG715V} experiments.

1. A synthetic methylmalonyl-CoA mutase polypeptide comprising an amino acid sequence comprising, in order from the N-terminus: a methylmalonyl-CoA mutase mitochondrial leader amino acid sequence, a tag amino acid sequence, and a methylmalonyl-CoA mutase mature amino acid sequence.

2. The synthetic polypeptide of claim 1, wherein the tag amino acid sequence is flanked by at least one linker amino acid sequence.

3. The synthetic polypeptide of claim 1, wherein the methylmalonyl-CoA mutase mitochondrial leader amino acid sequence comprises a human or a mouse mitochondrial leader amino acid sequence selected from the group consisting of SEQ ID NO:1, SEQ ID NO:2, and an amino acid sequence having at least about 95% identity thereto, and having substantially identical activity to the methylmalonyl-CoA mutase mitochondrial leader amino acid sequence.

4. The synthetic polypeptide of claim 2, wherein the linker amino acid sequence comprises an amino acid sequence of from 1 to about 15 amino acids which comprises only amino acids selected from the group consisting of serine (S), alanine (A), lysine (K), tyrosine (Y), threonine (T), phenylalanine (F), and glycine (G).

5. The linker amino acid sequence of claim 4, wherein the linker amino acid sequence is selected from the group consisting of MSYY (SEQ ID NO: 3), SKEFGT (SEQ ID NO: 4), GG (SEQ ID NO: 5), GGSS (SEQ ID NO: 6), and G (SEQ ID NO: 7).

6. The synthetic polypeptide of claim 1, wherein the amino acid tag is selected from the group consisting of a 3xFLAG tag, an HA tag, a V5 tag, a Myc-tag, a poly-HIS tag, a VSV tag, an Xpress tag, an isopeptag, and a spytag.

7. The synthetic polypeptide of claim 6, wherein the amino acid tag is selected from the group consisting of SEQ ID NO:8, SEQ ID NO:9, SEQ ID NO:10, SEQ ID NO:11, SEQ ID NO:12, SEQ ID NO:13, SEQ ID NO:14, SEQ ID NO:15, and SEQ ID NO:16.

8. The synthetic polypeptide of claim 6, wherein the amino acid tag is selected from the group consisting of SEQ ID NO:8, SEQ ID NO:9, and SEQ ID NO:10.

9. The synthetic polypeptide of claim 1, wherein the synthetic polypeptide comprises an amino acid sequence selected from the group consisting of SEQ ID NO:36, SEQ ID NO:37, and SEQ ID NO:38.

10. The synthetic polypeptide of claim 1, wherein the methylmalonyl-CoA mutase mature amino acid sequence is selected from the group consisting of SEQ ID NO:17, SEQ ID NO:18, and an amino acid sequence having at least about 95% identity thereto, and having substantially identical activity to the methylmalonyl-CoA mutase mature amino acid sequence.

11. The synthetic polypeptide of claim 1, wherein the polypeptide is selected from the group consisting of the amino acid sequence of SEQ ID NO:19, SEQ ID NO:20, SEQ ID NO:21, SEQ ID NO:22, SEQ ID NO:23, SEQ ID NO:24, SEQ ID NO:25, SEQ ID NO:26, and an amino acid sequence having at least about 95% identity thereto, and having substantially identical activity to the MUT mature amino acid sequence.

12. A synthetic methylmalonyl-CoA mutase polynucleotide which encodes for the polypeptide of claim 1.

13. The polynucleotide of claim 12, wherein said polynucleotide is selected from the group consisting of SEQ ID NO:27, SEQ ID NO:28, SEQ ID NO:29, SEQ ID NO:30, and a nucleic acid sequence having at least about 95% identity thereto, wherein the synthetic polynucleotide encodes for a polypeptide that has substantially identical activity to WT methylmalonyl-CoA mutase.

14. The polynucleotide of claim 12, wherein said polynucleotide comprises a polynucleotide that is selected from the group consisting of SEQ ID NO:31, SEQ ID NO:32, SEQ ID NO:33, SEQ ID NO:34, SEQ ID NO:35, wherein the polynucleotide further comprises a polynucleotide encoding a tag flanked by linkers, wherein the tag and linkers are inserted between nucleotides 96 and 97, and a polynucleotide sequence having at least about 95% identity thereto, wherein the synthetic polynucleotide encodes for a polypeptide that has substantially identical activity to WT methylmalonyl-CoA mutase.

15. The polynucleotide of claim 14, wherein the tag comprises a polynucleotide encoding the polypeptide selected from the group consisting of SEQ ID NO:8, SEQ ID NO:9, SEQ ID NO:10, SEQ ID NO:11, SEQ ID NO:12, SEQ ID NO:13, SEQ ID NO:14, SEQ ID NO:15, and SEQ ID NO:16, and the linkers comprise at least one polynucleotide encoding the polypeptide selected from the group consisting of MSYY (SEQ ID NO: 3), SKEFGT (SEQ ID NO: 4), GG (SEQ ID NO: 5), GGSS (SEQ ID NO: 6), and G (SEQ ID NO: 7); or the tag and linkers comprise a polynucleotide selected from the group consisting of SEQ ID NO:36, SEQ ID NO:37, and SEQ ID NO:38.

16. A synthetic polynucleotide selected from the group consisting of SEQ ID NO:27, SEQ ID NO:28, SEQ ID NO:29, SEQ ID NO:30, and a nucleic acid sequence having at least about 95% identity thereto, wherein the synthetic polynucleotide encodes for a polypeptide that has substantially identical activity to WT methylmalonyl-CoA mutase.

17. The synthetic polynucleotide of claim 12, wherein said nucleic acid sequence is a DNA sequence.

18. The synthetic polynucleotide of claim 12, wherein said nucleic acid sequence is a RNA sequence.

19. A recombinant expression vector comprising the polynucleotide of claim 12.

20. A composition comprising the synthetic polypeptide of claim 1 and a pharmaceutically acceptable carrier.

21. A composition comprising the synthetic polynucleotide of claim 12 and a pharmaceutically acceptable carrier.

22. A method of treating a disease or condition mediated by methylmalonyl-CoA mutase, the method comprising administering to a subject in need thereof, a therapeutic amount of a polynucleotide according to claim 12.

23. The method of claim 22, wherein the polynucleotide is expressed in the subject.

24. The method of claim 22, wherein the polynucleotide selected from the group consisting of SEQ ID NO:31, SEQ ID NO:32, SEQ ID NO:33, SEQ ID NO:34, SEQ ID NO:35, wherein the polynucleotide further comprises a polynucleotide encoding a tag flanked by linkers inserted between nucleotides 96 and 97, and a polynucleotide sequence having at least about 95% identity thereto, wherein the synthetic polynucleotide encodes for a polypeptide that has substantially identical activity to WT methylmalonyl-CoA mutase.

25. The method of claim 22, wherein the disease or condition is methylmalonic acidemia (MMA).

26. The method of claim 22, wherein the polynucleotide is inserted into a cell of the subject via genome editing on the cell of the subject using a nuclease selected from the group of zinc finger nucleases (ZFNs), transcription activator-like effector nucleases (TALENs), the clustered regularly interspaced short palindromic repeats (CRISPER/cas system) and meganuclease re-engineered homing endonucleases on a cell from the subject.

27. A method for detecting or tracking the expression of a synthetic polynucleotide of claim 11, comprising administering the synthetic polynucleotide in an expression vector to a subject, obtaining a sample of the patient's tissue, and determining the level of expression of the synthetic polynucleotide in the patient's tissue.


SEQUENCE LISTING (38 sequences)

SEQ ID NO: 1 - protein, 30 aa, Mus musculus
MLRAKNQLFLLSPHYLKQLNIPSASRWKRL

SEQ ID NO: 2 - protein, 32 aa, Homo sapiens
MLRAKNQLFLLSPHYLRQVKESSGSRLIQQRL

SEQ ID NO: 3 - protein, 4 aa, Artificial Sequence
  LINKING SEQUENCE
MSYY

SEQ ID NO: 4 - protein, 6 aa, Artificial Sequence
  LINKING SEQUENCE
SKEFGT

SEQ ID NO: 5 - protein, 2 aa, Artificial Sequence
  LINKING SEQUENCE
GG

SEQ ID NO: 6 - protein, 4 aa, Artificial Sequence
  LINKING SEQUENCE
GGSS

SEQ ID NO: 7 - protein, 1 aa, Artificial Sequence
  LINKING SEQUENCE
G

SEQ ID NO: 8 - protein, 27 aa, Artificial Sequence
  3X FLAG TAG
DYKDDDDKGDYKDDDDKGDYKDDDDKG

SEQ ID NO: 9 - protein, 9 aa, Artificial Sequence
  HA TAG
YPYDVPDYA

SEQ ID NO: 10 - protein, 14 aa, Artificial Sequence
  V5 TAG
GKPIPNPLLGLDST

SEQ ID NO: 11 - protein, 10 aa, Artificial Sequence
  MYC-TAG
EQKLISEEDL

SEQ ID NO: 12 - protein, 6 aa, Artificial Sequence
  HIS TAG
HHHHHH

SEQ ID NO: 13 - protein, 11 aa, Artificial Sequence
  Vsv tag
YTDIEMNRLGK

SEQ ID NO: 14 - protein, 8 aa, Artificial Sequence
  Xpress tag
DLYDDDDK

SEQ ID NO: 15 - protein, 16 aa, Artificial Sequence
  ISOPEPTAG
TDKDMTITFTNKKDAE

SEQ ID NO: 16 - protein, 13 aa, Artificial Sequence
  SPYTAG
AHIVMVDAYKPTK

SEQ ID NO: 17 - protein, 718 aa, Mus musculus
LHQQQPLHPEWAVLAKKQLKGKNPEDLIWHTPEGISIKPLYSRADTLDLPEELPGVKPFTRGPYPTMYTYRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLATFIVTGEEQGVPKEKLTGTIQNDILKEFMVRNTYIFPPEPSMKIIADIFQYTAQHMPKFNSISISGYHMQEAGADAILELAYTIADGLEYCRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMESLTNDVYEAALKLIYEVEEMGGMAKAVAEGIPKLRIEECAARRQARIDSGSEVIVGVNKYQLEKEDSVEVLAIDNTSVRKKQIEKLKKIKSSRDQALAEQCLSALTQCAASGDGNILALAVDAARARCTVGEITDALKKVFGEHKANDRMVSGAYRQEFGESKEITSAIKRVNKFMEREGRRPRLLVAKMGQDGHDRGAKVIATGFADLGFDVDIGPLFQTPREVAQQAVDADVHAVGVSTLAAGHKTLVPELIKELTALGRPDILVMCGGVIPPQDYEFLYEVGVSNVFGPGTRIPRAAVQVLDDIEKCLAEKQQSV

SEQ ID NO: 18 - protein, 718 aa, Homo sapiens
LHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 19 - protein, 772 aa, Artificial Sequence
  Expressed protein V5 MUT MURINE
MLRAKNQLFLLSPHYLKQLNIPSASRWKRLMSYYGKPIPNPLLGLDSTSKEFGTLHQQQPLHPEWAVLAKKQLKGKNPEDLIWHTPEGISIKPLYSRADTLDLPEELPGVKPFTRGPYPTMYTYRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLATFIVTGEEQGVPKEKLTGTIQNDILKEFMVRNTYIFPPEPSMKIIADIFQYTAQHMPKFNSISISGYHMQEAGADAILELAYTIADGLEYCRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMESLTNDVYEAALKLIYEVEEMGGMAKAVAEGIPKLRIEECAARRQARIDSGSEVIVGVNKYQLEKEDSVEVLAIDNTSVRKKQIEKLKKIKSSRDQALAEQCLSALTQCAASGDGNILALAVDAARARCTVGEITDALKKVFGEHKANDRMVSGAYRQEFGESKEITSAIKRVNKFMEREGRRPRLLVAKMGQDGHDRGAKVIATGFADLGFDVDIGPLFQTPREVAQQAVDADVHAVGVSTLAAGHKTLVPELIKELTALGRPDILVMCGGVIPPQDYEFLYEVGVSNVFGPGTRIPRAAVQVLDDIEKCLAEKQQSV

SEQ ID NO: 20 - protein, 742 aa, Artificial Sequence
  Mature expressed protein V5 MUT  MURINE
MSYYGKPIPNPLLGLDSTSKEFGTLHQQQPLHPEWAVLAKKQLKGKNPEDLIWHTPEGISIKPLYSRADTLDLPEELPGVKPFTRGPYPTMYTYRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLATFIVTGEEQGVPKEKLTGTIQNDILKEFMVRNTYIFPPEPSMKIIADIFQYTAQHMPKFNSISISGYHMQEAGADAILELAYTIADGLEYCRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMESLTNDVYEAALKLIYEVEEMGGMAKAVAEGIPKLRIEECAARRQARIDSGSEVIVGVNKYQLEKEDSVEVLAIDNTSVRKKQIEKLKKIKSSRDQALAEQCLSALTQCAASGDGNILALAVDAARARCTVGEITDALKKVFGEHKANDRMVSGAYRQEFGESKEITSAIKRVNKFMEREGRRPRLLVAKMGQDGHDRGAKVIATGFADLGFDVDIGPLFQTPREVAQQAVDADVHAVGVSTLAAGHKTLVPELIKELTALGRPDILVMCGGVIPPQDYEFLYEVGVSNVFGPGTRIPRAAVQVLDDIEKCLAEKQQSV

SEQ ID NO: 21 - protein, 775 aa, Artificial Sequence
  expressed protein V5 mut HUMAN
MLRAKNQLFLLSPHYLRQVKESSGSRLIQQRLMSYYGKPIPNPLLGLDSTSKEFGTLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 22 - protein, 743 aa, Artificial Sequence
  mature expressed protein V5 mut human
MSYYGKPIPNPLLGLDSTSKEFGTLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 23 - protein, 765 aa, Artificial Sequence
  SYNMUT4-HA
MLRAKNQLFLLSPHYLRQVKESSGSRLIQQRLGGSSYPYDVPDYAGGLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 24 - protein, 733 aa, Artificial Sequence
  SYNMUT-4 HA  MATURE PROTEIN
GGSSYPYDVPDYAGGLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 25 - protein, 782 aa, Artificial Sequence
  SYNMUT4-3XFLAG
MLRAKNQLFLLSPHYLRQVKESSGSRLIQQRLGGSSDYKDDDDKGDYKDDDDKGDYKDDDDKGGLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 26 - protein, 750 aa, Artificial Sequence
  SYNMUT4-3XFLAG MATURE PROTEIN
GGSSDYKDDDDKGDYKDDDDKGDYKDDDDKGGLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 27 - DNA, 2328 bp, Artificial Sequence
  Engineered V5 MUT human SYNMUT1
atgctgagagccaaaaaccagctgttcctgctgagcccccactatctgagacaggtcaaagaaagttccgggagtagactgatccagcagagactgatgtcctactacggcaagcccatccccaaccccctgctgggcctggactccacctccaaggagttcggcaccctactgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 28 - DNA, 2319 bp, Artificial Sequence
  CDS for V5 MUT MURINE
atgttgagagctaagaatcaactttttttgctatcgccccattacctgaagcagctaaacattccatcagcttccagatggaaacgccttatgtcctactacggcaagcccatccccaaccccctgctgggcctggactccacctccaaggagttcggcaccctacaccagcagcaaccccttcacccagaatgggctgtactggccaaaaagcagctgaaaggcaaaaacccagaggaccttatatggcacaccccagaagggatctctataaagcccttatattccagggcagatactctggacttacccgaagaacttccaggagtgaagccattcacacggggaccatatcctaccatgtatacctataggccctggaccatccgtcagtatgcaggctttagtactgtggaagaaagcaataaattctataaggacaatattaaggctggtcagcaggggttgtctgttgcctttgacttggcaacacatcgtggttatgattcagacaaccccagagttcgtggagatgttggaatggctggagttgctattgacactgtagaagacaccaaaatcctatttgatggcattcctttagaaaaaatgtccgtttccatgactatgaacggagctgtcatcccagtcctggcaacatttatagtaactggggaagagcaaggtgtgccgaaggagaagctcactggcacaattcagaatgacatcctaaaggagtttatggtcagaaatacttatatttttcccccagagccatcgatgaaaattattgctgacattttccaatacacagcacagcacatgccaaaatttaattccatttcgattagcgggtaccatatgcaggaagcaggagctgatgccattttagaactggcctataccatcgcagatgggttggagtactgcagaactggactccaggctggactcacaattgatgaatttgcaccaaggttgtctttcttctgggggattggaatgaacttctacatggaaatagccaagatgcgagctgggagaagactgtgggctcacttaatagaaaaaatgttccagcctaaaaactctaaatctcttcttctaagagcacactgccagacatcggggtggtcacttactgagcaggatccttacaataacattgtccgcactgcaatcgaagccatggcagctgtgtttggaggaacccagtctttgcatacgaactcttttgatgaagcgctgggtttgcccactgtgaaaagtgcccggattgcccggaacacacagatcatcattcaggaggaatctgggatccccaaagtggcggatccttggggagggtcgtacatgatggagtcgctcacaaatgacgtttatgaggctgctctgaagttgatatatgaagttgaagaaatgggtggaatggccaaagctgtagctgaaggaatccctaaacttcgcattgaagaatgtgctgcccgaagacaagctagaatagattctggttctgaggtaattgttggagtaaataagtatcagttggaaaaagaagactctgtggaggtcctggccattgacaacacttcagtgcgtaagaagcagattgaaaaactcaagaagattaaatccagcagggatcaagctttggctgagcagtgtctcagtgcacttacccagtgtgctgccagtggagatggcaatattctggctctggcagtggatgcagctcgtgcaagatgtacagttggagaaatcacggatgccttgaaaaaagtatttggtgagcataaagctaatgatcgtatggtgagtggagcatatcggcaggagtttggagaaagtaaagagatcacatctgccatcaagagagttaataaattcatggaacgtgaaggtcgcagacctcgtcttcttgtggcaaaaatgggacaagatggccatgacaggggagccaaggtcattgctacaggatttgctgatcttggttttgatgtggacataggccctctttttcagactccccgtgaagtggcgcagcaagctgtggatgcagatgtccatgctgtgggtgtcagcacacttgctgctggtcataaaaccctcgttcctgagcttatcaaagaactcactgccctggggcggccagatatccttgtcatgtgtgggggcgtgattccaccacaggattatgaatttctgtatgaagttggtgtttccaacgtctttggtcctggaacccggattcctagagctgctgtccaagtgcttgatgatattgagaagtgtttggcagagaagcagcagtctgtgtaa

SEQ ID NO: 29 - DNA, 2301 bp, Artificial Sequence
  CDS FOR SYNMUT4-HA TAG
atgcttcgcgccaagaaccaactgttcctgctgtccccccactacctccgacaagtcaaggagagctcgggaagccgcctgattcagcagcggctggggggtagcagctacccctacgatgtgcctgattacgccggtggactgcaccagcagcagcccctgcatccggaatgggcagcgttggcaaagaagcagctgaagggaaagaaccctgaggacctgatctggcacaccccggagggaatctcgatcaagccactgtactccaaaagggacaccatggacttgcctgaagaacttccgggcgtgaagccttttacccgggggccatacccaacaatgtacactttccgcccctggaccatcagacagtacgccggtttctccaccgtcgaagaatccaacaagttctataaggacaacatcaaggccgggcagcagggactgagcgtcgcgtttgacctggcaacccatcgcggctacgactccgacaaccctcgcgtgcggggggacgtgggaatggccggagtggctatcgacaccgtggaggacaccaagattctcttcgacggaatcccgctggaaaagatgtcggtgtccatgaccatgaatggcgccgtgatcccggtgctcgcgaacttcatcgtgacgggagaggaacagggagtgccgaaagagaagctgaccgggactattcagaatgacatcctcaaggagttcatggtccgcaacacttacattttccctcctgaaccctcgatgaagatcatcgctgacatcttcgagtacaccgcgaagcacatgccgaagttcaactcgatctccatctcgggctaccacatgcaggaggccggggccgacgccattctcgaactggcgtacactctggcggatggtctggaatactcacgcaccggactgcaggccggactgacaatcgacgagttcgccccgaggctgtccttcttctggggcattgggatgaacttctatatggaaatcgcgaagatgagagctggaaggcggctgtgggcgcacctgatcgagaagatgttccagcccaagaacagcaaaagccttctcctccgcgcccactgccaaacttccggctggtcactgaccgagcaggatccgtacaacaacattgtccggactgccattgaggccatggccgctgtgttcggaggcactcagtccctccacactaactccttcgacgaggccctgggtctgccgaccgtgaagtccgcccggatagccagaaatactcaaatcattatccaggaggaaagcggaatccccaaggtcgccgacccttggggaggatcttacatgatggagtgtttgaccaatgacgtctacgacgccgccctgaagctcattaacgaaatcgaagagatgggcggaatggccaaggccgtggctgagggcatcccgaagctgagaatcgaggaatgcgccgcccggagacaggcccgcattgatagcggcagcgaggtcattgtgggcgtgaacaagtaccagcttgaaaaggaggacgccgtggaagtgctggcaatcgataacacctccgtgcgcaaccggcagatcgaaaagctcaagaagattaagtcctcacgggaccaggcactggcggagagatgcctcgccgcgctgaccgaatgcgctgcctcgggagatggcaacattctggccctggcagtggacgcctctcgggctcggtgcactgtgggggagatcaccgacgccctcaagaaagtgttcggtgaacataaggccaacgaccggatggtgtccggagcgtaccgccaggaatttggcgaatcaaaggaaatcacgtccgcaatcaagagggtgcacaaattcatggaacgggagggcagacggcccagactgctcgtggctaaaatgggacaagatggtcacgaccgcggcgccaaggtcatcgcgactggcttcgccgatctcggattcgacgtggacatcggacctctgtttcaaactccccgggaagtggcccagcaggccgtggacgcggacgtgcatgccgtcgggatctcaaccctggcggccggccataagaccctggtgccggaactgatcaaggagctgaactcgctcggccgccccgacatcctcgtgatgtgtggcggagtgattccgccacaagactacgagttcctgttcgaagtcggggtgtccaacgtgttcggtcccggaaccagaatcccgaaggctgcggtccaagtgctggatgatattgagaagtgccttgagaaaaagcaacagtcagtgtgatag

SEQ ID NO: 30 - DNA, 2349 bp, Artificial Sequence
  synMUT4_3xFLAG
atgcttcgcgccaagaaccaactgttcctgctgtccccccactacctccgacaagtcaaggagagctcgggaagccgcctgattcagcagcggctggggggtagcagcgactacaaggatgacgatgacaagggagattacaaggacgatgacgataagggcgattacaaagatgacgacgacaagggtggactgcaccagcagcagcccctgcatccggaatgggcagcgttggcaaagaagcagctgaagggaaagaaccctgaggacctgatctggcacaccccggagggaatctcgatcaagccactgtactccaaaagggacaccatggacttgcctgaagaacttccgggcgtgaagccttttacccgggggccatacccaacaatgtacactttccgcccctggaccatcagacagtacgccggtttctccaccgtcgaagaatccaacaagttctataaggacaacatcaaggccgggcagcagggactgagcgtcgcgtttgacctggcaacccatcgcggctacgactccgacaaccctcgcgtgcggggggacgtgggaatggccggagtggctatcgacaccgtggaggacaccaagattctcttcgacggaatcccgctggaaaagatgtcggtgtccatgaccatgaatggcgccgtgatcccggtgctcgcgaacttcatcgtgacgggagaggaacagggagtgccgaaagagaagctgaccgggactattcagaatgacatcctcaaggagttcatggtccgcaacacttacattttccctcctgaaccctcgatgaagatcatcgctgacatcttcgagtacaccgcgaagcacatgccgaagttcaactcgatctccatctcgggctaccacatgcaggaggccggggccgacgccattctcgaactggcgtacactctggcggatggtctggaatactcacgcaccggactgcaggccggactgacaatcgacgagttcgccccgaggctgtccttcttctggggcattgggatgaacttctatatggaaatcgcgaagatgagagctggaaggcggctgtgggcgcacctgatcgagaagatgttccagcccaagaacagcaaaagccttctcctccgcgcccactgccaaacttccggctggtcactgaccgagcaggatccgtacaacaacattgtccggactgccattgaggccatggccgctgtgttcggaggcactcagtccctccacactaactccttcgacgaggccctgggtctgccgaccgtgaagtccgcccggatagccagaaatactcaaatcattatccaggaggaaagcggaatccccaaggtcgccgacccttggggaggatcttacatgatggagtgtttgaccaatgacgtctacgacgccgccctgaagctcattaacgaaatcgaagagatgggcggaatggccaaggccgtggctgagggcatcccgaagctgagaatcgaggaatgcgccgcccggagacaggcccgcattgatagcggcagcgaggtcattgtgggcgtgaacaagtaccagcttgaaaaggaggacgccgtggaagtgctggcaatcgataacacctccgtgcgcaaccggcagatcgaaaagctcaagaagattaagtcctcacgggaccaggcactggcggagagatgcctcgccgcgctgaccgaatgcgctgcctcgggagatggcaacattctggccctggcagtggacgcctctcgggctcggtgcactgtgggggagatcaccgacgccctcaagaaagtgttcggtgaacataaggccaacgaccggatggtgtccggagcgtaccgccaggaatttggcgaatcaaaggaaatcacgtccgcaatcaagagggtgcacaaattcatggaacgggagggcagacggcccagactgctcgtggctaaaatgggacaagatggtcacgaccgcggcgccaaggtcatcgcgactggcttcgccgatctcggattcgacgtggacatcggacctctgtttcaaactccccgggaagtggcccagcaggccgtggacgcggacgtgcatgccgtcgggatctcaaccctggcggccggccataagaccctggtgccggaactgatcaaggagctgaactcgctcggccgccccgacatcctcgtgatgtgtggcggagtgattccgccacaagactacgagttcctgttcgaagtcggggtgtccaacgtgttcggtcccggaaccagaatcccgaaggctgcggtccaagtgctggatgatattgagaagtgccttgagaaaaagcaacagtcagtgtga

SEQ ID NO: 31 - DNA, 2253 bp, Artificial Sequence
  HUMAN MUT
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 32 - DNA, 2253 bp, Artificial Sequence
  SYNMUT1
atgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 33 - DNA, 2253 bp, Artificial Sequence
  SynMUT2
atgctgcgagcgaaaaatcagctttttctgttgagcccacactacctgaggcaggttaaagaatccagcgggagccggctgattcagcagcgactgctccaccagcagcagcctttgcatcccgaatgggctgctttggcgaagaagcagctcaaggggaagaaccctgaagatcttatttggcacaccccagagggcatcagcatcaagcctttgtattccaaaagggacaccatggatctgcctgaagaattgcccggggtcaaaccattcacacgggggccatatccaaccatgtacaccttccggccatggactatcagacagtatgcaggctttagcactgtcgaggaatccaataagttctataaagacaatatcaaagctggccagcaaggtctgtccgtggcattcgatctggctacacatagaggttatgattctgacaatccaagagtacggggagacgtcggaatggcgggagttgccattgacacagtggaggacaccaagatacttttcgatgggattccattggagaaaatgtctgtgtcaatgacgatgaacggcgctgtgattcccgttttggcgaacttcatcgtcaccggggaagagcagggcgtcccgaaggaaaagctcaccgggacaatccaaaacgacattcttaaagaattcatggtgagaaatacctacatctttcctcctgagccttccatgaagatcatcgcggacatctttgaatacacggctaaacacatgcctaaatttaactcaatcagcataagcgggtaccacatgcaggaggccggcgctgacgctatacttgagctcgcatataccctggcagatggactggaatactcaaggaccgggctccaggctggactgacaatcgacgagtttgccccccgactcagttttttctggggtatcgggatgaatttctacatggagatagcgaagatgagggcgggcagacggctttgggcgcatctgatcgagaaaatgttccagcccaagaattcaaagagtctgctgctgagagcccactgccagacctcaggctggagcctgactgaacaggacccatacaacaacattgttagaaccgccatcgaggcgatggcagcggttttcggtgggacacagtcattgcacactaactcatttgacgaagccctcggtctgcctaccgtgaagtcagctcggatcgctaggaacacacagatcatcatccaggaggagagtggcatcccaaaagtcgccgatccttggggaggaagttacatgatggaatgcctcacgaatgacgtatacgatgccgcactcaagctgattaacgagatcgaggaaatgggaggcatggcaaaagctgtcgccgagggcattccaaagctgcgcatagaggagtgtgccgcccgaagacaggcccgcattgactccggctctgaggtgatagtgggcgttaataaatatcagctagagaaggaagacgccgtcgaagttctggcgatagataatacctctgtgcgaaatagacagattgagaaactgaagaagatcaagtcaagccgagaccaggccttggccgagaggtgtctggcagccctcactgagtgcgcggcatctggggacggcaacatattggcacttgccgtcgatgcctccagggcccgatgtacggtcggcgaaattaccgatgccctcaagaaggtttttggcgagcacaaggctaacgacaggatggttagtggagcatacagacaggagtttggcgaaagcaaggaaattacttccgcgattaaaagagtgcacaaattcatggaacgggagggtaggcgaccgaggctcctcgttgccaaaatgggtcaggacggccacgaccggggcgccaaggttatcgctaccggtttcgctgacctgggcttcgatgtggatatcggaccactgtttcaaacccccagagaagttgcccaacaagccgttgacgctgacgtacacgctgtaggcatctccactctcgccgccgggcataagactctcgtcccagagctgataaaggagcttaacagcctcggaagacccgacatcctggttatgtgcggtggagtgattccgccgcaggattacgaattcctcttcgaagtaggagtgtcaaacgtgttcggcccaggcactcggatacccaaggctgccgttcaggtgcttgacgacattgaaaaatgtctggagaagaagcaacaatctgtataa

SEQ ID NO: 34 - DNA, 2253 bp, Artificial Sequence
  SynMUT3
atgttgagggctaaaaaccagctctttctgttgagtccacactaccttaggcaagtgaaggaatctagcggtagcaggctgatccagcagcgcctgctgcaccagcagcagcccctgcaccctgagtgggctgcattggcaaagaaacaactgaagggtaaaaatcctgaagatctgatttggcacacaccggaggggatttccataaaacctctctactctaaacgcgatactatggatctgcccgaggaattgccaggagtgaaaccctttacaagggggccctaccccactatgtacacgttcagaccctggactatacgccagtatgccggattttctaccgttgaggaatccaacaagttttataaggacaacatcaaagccgggcagcagggactgtcagtggcatttgatctcgccacccaccgcgggtacgactccgacaacccaagagtccgcggtgacgtcggcatggcaggggttgccattgacacagtagaggatactaaaattttgtttgatgggatccccctagagaagatgtccgtgtctatgacgatgaacggcgcggtaatcccagtgcttgccaacttcatagtcacaggggaagagcagggcgtaccaaaggagaagctcacaggaacaatccaaaatgacattctgaaggaattcatggtgagaaatacttatatctttcctcccgagccctctatgaagattattgccgacatttttgaatacaccgcaaaacatatgcccaagttcaattccatatctattagtggataccacatgcaagaagctggggctgatgcaatacttgagcttgcctacaccctggccgacggactggagtattctcgcactggcctgcaagccgggctgacaattgacgagttcgccccacgccttagcttcttctggggcatcggcatgaatttctatatggagatcgcaaagatgagagcagggcggcgcttgtgggcccatctgatcgaaaagatgtttcagcctaagaatagtaagagcctgctcctgcgggctcactgtcagacgtcaggctggagcctcacagagcaggatccttacaataacatcgtccggactgctattgaggcgatggctgcagtattcggaggaacacaaagcctgcacactaattctttcgatgaggctttggggctccctaccgtgaagtcagccagaattgcaagaaacacccaaataatcatccaagaagaatcagggatcccaaaagttgccgacccctggggaggaagttatatgatggagtgcctgaccaatgacgtctacgacgccgctttgaagctgattaacgagattgaagagatgggcggaatggccaaggcggtcgctgagggcattccgaaactgcgcatagaggagtgtgctgctcgcaggcaggccagaattgattccggttccgaagtgatcgtgggggttaataagtatcaactggaaaaagaggacgctgtcgaagtcctcgcaatcgataataccagcgttagaaaccgacaaattgagaagctgaaaaagatcaaaagttcaagggaccaggccttggctgagcggtgtctcgccgcactgaccgaatgtgccgccagcggcgatggtaacatcctcgccctcgctgtggacgcttccagagcccggtgcaccgtgggcgaaattacggacgcgctgaaaaaagtctttggcgaacacaaggccaatgatagaatggtgagtggcgcctataggcaggagttcggcgagagtaaagaaataacatccgccatcaagagggtccacaaatttatggagcgggaaggacgcagacctagacttctcgtggccaaaatgggtcaggacggtcatgaccggggagccaaagtcatcgcaacgggcttcgccgatttggggtttgacgtggatatcggtcccttgtttcaaacccccagggaggtggctcagcaggctgtggacgctgacgtccacgcagtgggcatttctacactggcagccgggcacaagacgttggtgccagaactgatcaaagagttgaacagcctgggacgccctgacatcctggtaatgtgcggtggggtaatccccccccaagactacgagttccttttcgaagtgggtgtttctaacgtgttcggacctggaacaagaatccctaaggcggcagtgcaggtgcttgacgatatcgagaagtgcctggagaaaaagcaacaatccgtttaa

SEQ ID NO: 35 - DNA, 2253 bp, Artificial Sequence
  SynMUT4
atgcttcgcgccaagaaccaactgttcctgctgtccccccactacctccgacaagtcaaggagagctcgggaagccgcctgattcagcagcggctgctgcaccagcagcagcccctgcatccggaatgggcagcgttggcaaagaagcagctgaagggaaagaaccctgaggacctgatctggcacaccccggagggaatctcgatcaagccactgtactccaaaagggacaccatggacttgcctgaagaacttccgggcgtgaagccttttacccgggggccatacccaacaatgtacactttccgcccctggaccatcagacagtacgccggtttctccaccgtcgaagaatccaacaagttctataaggacaacatcaaggccgggcagcagggactgagcgtcgcgtttgacctggcaacccatcgcggctacgactccgacaaccctcgcgtgcggggggacgtgggaatggccggagtggctatcgacaccgtggaggacaccaagattctcttcgacggaatcccgctggaaaagatgtcggtgtccatgaccatgaatggcgccgtgatcccggtgctcgcgaacttcatcgtgacgggagaggaacagggagtgccgaaagagaagctgaccgggactattcagaatgacatcctcaaggagttcatggtccgcaacacttacattttccctcctgaaccctcgatgaagatcatcgctgacatcttcgagtacaccgcgaagcacatgccgaagttcaactcgatctccatctcgggctaccacatgcaggaggccggggccgacgccattctcgaactggcgtacactctggcggatggtctggaatactcacgcaccggactgcaggccggactgacaatcgacgagttcgccccgaggctgtccttcttctggggcattgggatgaacttctatatggaaatcgcgaagatgagagctggaaggcggctgtgggcgcacctgatcgagaagatgttccagcccaagaacagcaaaagccttctcctccgcgcccactgccaaacttccggctggtcactgaccgagcaggatccgtacaacaacattgtccggactgccattgaggccatggccgctgtgttcggaggcactcagtccctccacactaactccttcgacgaggccctgggtctgccgaccgtgaagtccgcccggatagccagaaatactcaaatcattatccaggaggaaagcggaatccccaaggtcgccgacccttggggaggatcttacatgatggagtgtttgaccaatgacgtctacgacgccgccctgaagctcattaacgaaatcgaagagatgggcggaatggccaaggccgtggctgagggcatcccgaagctgagaatcgaggaatgcgccgcccggagacaggcccgcattgatagcggcagcgaggtcattgtgggcgtgaacaagtaccagcttgaaaaggaggacgccgtggaagtgctggcaatcgataacacctccgtgcgcaaccggcagatcgaaaagctcaagaagattaagtcctcacgggaccaggcactggcggagagatgcctcgccgcgctgaccgaatgcgctgcctcgggagatggcaacattctggccctggcagtggacgcctctcgggctcggtgcactgtgggggagatcaccgacgccctcaagaaagtgttcggtgaacataaggccaacgaccggatggtgtccggagcgtaccgccaggaatttggcgaatcaaaggaaatcacgtccgcaatcaagagggtgcacaaattcatggaacgggagggcagacggcccagactgctcgtggctaaaatgggacaagatggtcacgaccgcggcgccaaggtcatcgcgactggcttcgccgatctcggattcgacgtggacatcggacctctgtttcaaactccccgggaagtggcccagcaggccgtggacgcggacgtgcatgccgtcgggatctcaaccctggcggccggccataagaccctggtgccggaactgatcaaggagctgaactcgctcggccgccccgacatcctcgtgatgtgtggcggagtgattccgccacaagactacgagttcctgttcgaagtcggggtgtccaacgtgttcggtcccggaaccagaatcccgaaggctgcggtccaagtgctggatgatattgagaagtgccttgagaaaaagcaacagtcagtgtga

SEQ ID NO: 36 - protein, 15 aa, Artificial Sequence
  Linker-tag-linker
GGSSYPYDVPDYAGG

SEQ ID NO: 37 - protein, 32 aa, Artificial Sequence
  Linker-tag-linker
GGSSDYKDDDDKGDYKDDDDKGDYKDDDDKGG

SEQ ID NO: 38 - protein, 24 aa, Artificial Sequence
  Linker-tag-linker
MSYYGKPIPNPLLGLDSTSKEFGT